(19) United States
(12) Patent Application Publication
(10) Pub. No.: US 2006/0117398 A1
(43) Pub. Date: Jun. 1, 2006

(54) Suppression of endogenous immunoglobulin expression

(76) Inventors: Roland Buelow, Palo Alto, CA (US); Josef Platzer, Geretsried (DE)

Correspondence Address: HELLER EHRMAN LLP, 275 MIDDLEFIELD ROAD, MENLO PARK, CA, 94025-3506, US

(21) Appl. No.: 11/255,820

(22) Filed: Oct. 21, 2005

Related U.S. Application Data
(60) Provisional application No. 60/621,228, filed on Oct. 22, 2004.

(51) Int. Cl. A01K 67/027; C12N 15/86

(52) U.S. Cl. 800/14; 800/15; 800/16; 800/17; 800/18; 800/19; 435/456

(57) ABSTRACT

The invention provides a novel approach for the suppression of endogenous antibody expression in non-human transgenic animals genetically engineered to express one or several human or humanized immunoglobulin transloci. Endogenous immunoglobulin expression in transgenic non-human animals is suppressed by selective expression of a suicide gene like a toxin only in B-cells expressing endogenous immunoglobulin but not in B-cells expressing human(ized) immunoglobulins. This method allows the dominant expression of transloci coding for humanized or human antibodies in the blood, milk and eggs of transgenic animals.

### CROSS-REFERENCE TO RELATED APPLICATIONS

This is a non-provisional application filed under 37 CFR 1.53(b), claiming priority under U.S.C. Section 119(e) to U.S. Provisional Patent Application Ser. No. 60/621,228 filed Oct. 22, 2004.

### FIELD OF THE INVENTION

This invention relates to a method to suppress expression of endogenous immunoglobulin in non-human transgenic animals by selective expression of a suicide gene in B-cells expressing endogenous immunoglobulin but not in B-cells expressing an exogenous immunoglobulin or immunoglobulin chain, such as human or humanized immunoglobulins or immunoglobulin chains. This method allows the dominant expression of human or humanized antibodies, for example in the blood, milk or eggs of the transgenic non-human animals.

### BACKGROUND ART

The generation of mice expressing human-mouse chimeric antibodies has been described by Pluschke et al., Journal of Immunological Methods 215: 27-37 (1998). The generation of mice expressing human immunoglobulin polypeptides has been described by Neuberger et al., Nature 338: 350-2 (1989); Lonberg et al., Int. Rev. Immunol. 13(1):65-93 (1995); and Bruggemann et al., Curr. Opin. Biotechnol., 8(4): 455-8 (1997) and. Generation of transgenic mice using a BAC clone has been described by Yang et al., Nat. Biotechnol. 15: 859-65 (1997). The generation of cows expressing human antibodies has been described by Kuroiwa et al., Nature Biotech 20(9): 889-894 (2002).

Transgenesis in animals has been described by Wall R J, Theriogenology 57(1): 189-201 (2002). The generation of transgenic rabbits has been described by Fan, J. et al., Pathol Int. 49: 583-94 (1999); and Brem et al., Mol. Reprod. Dev. 44: 56-62 (1996). The production of transgenic chicken has been described by Etches et al., Methods in Molecular Biology 62: 433-450 (1997); and Pain et al., Cells Tissues Organs 165(3-4): 212-9 (1999); and Sherman et al., Nature Biotech 16:1050-1053 (1998).

Rabbits with impaired immunoglobulin expression have been described by Chen et al., J. Immunol. 150:2783-2793 (1993); and Lamoyi E, and Mage R G., J. Exp. Med. 162:1149-1160 (1985). A gamma-globulinemic chicken has been described by Frommel et al., J. Immunol. 105(1): 1-6 (1970); and Benedict et al., Adv. Exp. Med. Biol. 88(2): 197-205 (1977).

The cloning of animals from cells has been described by T. Wakayama et al., Nature 394:369-374 (1998); J. B. Cibelli et al., Science 280:1256-1258 (1998); J. B. Cibelli et al., Nature Biotechnology 16:642-646 (1998); A. E. Schnieke et al., Science 278: 2130-2133 (1997); and K. H. Campbell et al., Nature 380: 64-66 (1996). Nuclear transfer cloning of rabbits has been described by Stice et al., Biology of Reproduction 39: 657-664 (1988), Challah-Jacques et al., Cloning and Stem Cells 8(4):295-299 (2003).

The production of non-human transgenic animals expressing human(ized) immunoglobulin transloci and the production of antibodies from such transgenic animals have been described in detail in PCT Publication Nos. WO 92/03918, WO 02/12437, and in U.S. Pat. Nos. 5,545,807, 5,814,318; and 5,570,429. Homologous recombination for chimeric mammalian hosts is exemplified in U.S. Pat. No. 5,416,260. A method for introducing DNA into an embryo is described in U.S. Pat. No. 5,567,607. Maintenance and expansion of embryonic stem cells is described in U.S. Pat. No. 5,453,357.

Suicide genes using the toxin based approach have been described in Leong et al., Science, 220:515-7, (1983); Maxwell et al., Cancer Research, 46:4660-4664, (1986); Palmiter et al., Cell, 50:435-443, (1987); Maxwell et al., Cell, 51:4299-4304, (1991); Maxwell et al., Leukemia and Lymphoma, 7:457-462, (1992); Aguila et al., Proc. Natl. Acad. Sci., 92:10192-10196 (1995); Grieshammer et al., Developmental Biology, 197:234-247, (1998); Bartell et al., Biology of Reproduction, 63:409-416 (2000); Erlandsson et al., J. Exp. Med., 194:557-570 (2001); Lee et al., Human Gene Therapy, 13:533-542 (2002). Suicide genes using a non-toxic prodrug-enzyme approach have been described in (Methods in Molecular Medicine: Suicide Gene Therapy, Methods and Reviews, edited by Caroline J Springer, Humana Press, 2004).

The cleavage activities of viral proteins containing 2A peptide sequences have been described by Palmenberg et al., Virology 190:754-762 (1992), Ryan et al., J Gen Virol 72:2727-2732 (1991), Donnelly et al., J Gen Virol 82:1027-1041 (2001), Donnelly et al., J Gen Virol 82:1013-1025 (2001), Szymaczak et al., Nature Biotech 22(5):589-594 (2004).

Recombinases and their properties have been described by Kolb A F. Cloning Stem Cells 41: 65-80 (2002). Site-specific recombinases that recognize and catalyze homologous recombination between very specific sequences in two nucleic acids are known. For example, φC31 and R4 that belong to the integrase family of site-specific recombinases are known, Groth et al., Proc., Natl. Acad. Sci., 97: 5995-6000 (2000); Olivares et al., Nature Biotechnol., 20(11): 1124-8 (2002). Pseudo-attP sites are native in some genomes, including the human and mouse genome, Thyagarajan et al., Mol. and Cell. Biol., 21: 3926-3934 (2001). φC31 integrase mediated gene transfer of a large type VII collagen cDNA of 8.9 kb into primary progenitor patient skin cells in vitro has been reported Ortiz-Urda et al., Nature Medicine, 8:1166-1170 (2002). Use of φC31, TP901-1, and R4 phage integrases in the manipulation of transgenic mammals has been demonstrated by Hollis et al., Repro. Biol. and Endocrinol., 1:79 (2003).

Ablation of cells, including B-cells has been described by Erlandsson et al., J Exp Med 194(5):557-570 (2001), Maxwell et al., Cancer Research 51:4299-4304 (1991) and Palmiter et al., Cell 50:435-443 (1987).

### SUMMARY OF THE INVENTION

The present invention relates to a method for suppressing endogenous immunoglobulin production in transgenic animals. The method involves selectively expressing a suicide gene in B-cells expressing endogenous immunoglobulin but not not expressing the suicide gene in B-cells expressing human or humanized immunoglobulins. In particular, the invention concerns a method for suppressing expression of endogenous immunoglobulin loci in non-human transgenic animals containing one or several human or human(ized) immunoglobulin transloci. As a result, the human(ized) transloci are capable of undergoing gene rearrangement and mutational processes in the transgenic non-human animals to produce a diversified human(ized) antibody repertoire, substantially in the absence of endogenous immunoglobulin production.

In particular, the invention concerns a method for the selective suppression of endogenous immunoglobulin (Ig) production in B-cells of a non-human transgenic animal carrying an exogenous immunoglobulin translocus, comprising selectively expressing at least one suicide gene in B-cells producing an endogenous immunoglobulin of the non-human transgenic animal, but not in B-cells producing an exogenous immunoglobulin, whereby B cells producing the endogenous immunoglobulin are depleted, and production of the endogenous immunoglobulin is suppressed, without suppressing the production of the exogenous immunoglobulin. In a preferred embodiment, the exogenous immunoglobulin is a humanized immunoglobulin heavy and/or light chain sequence.

In a further aspect, the suicide gene introduced into the B-cells of the non-human transgenic animal is under the control of a B-cell specific promoter and is flanked by recombination sequences.

In yet another further aspect, the human(ized) immunoglobulin chain translocus is introduced into the B-cells of the non-human transgenic animal as part of an expression construct additionally encoding a recombinase recognizing said recombination sequences, wherein expression of the suicide gene is activated through expression of the recombinase in B-cells expressing the humanized immunoglobulin translocus.

In a certain aspect, the suicide gene is selected from the group consisting of a bacterial, fungal, insecticidal and plant toxins. In a preferred embodiment, the suicide gene is diphteria toxin chain A.

In another embodiment, the suicide gene is a prodrug converting enzyme. In one aspect of this embodiment, the prodrug converting enzyme is of non-mammalian origin. In a further aspect, the non-mammalian prodrug converting enzyme is selected from the group consisting of viral thymidine kinase (TK), bacterial cytosine deaminase (CD), bacterial carboxypeptidase G2 (CPG2), purine nucleotide phosphorylase (PNP), thymidine phosphorylase (TP), nitroreductase (NR), D-amino acid oxidase (DAAO), xanthine-guanine phosphoribosyl transferease (XGPRT), penicillin-G amidase (PGA), β-lactamase, multiple drug activation enzyme (MDAE), β-galactosidase (β-Gal), horseradish peroxidase (HRP) and deoxyribonucleotide kinase (DRNK).

In yet another embodiment, the prodrug converting enzyme is of human origin. In a further aspect, the human prodrug converting enzyme is selected from the group consisting of deoxycytidine kinase (dCK), carboxlesterases (CEs), carboxypeptidase A (CPA), β-glucuronidase (-Glu), and cytochrome P450 (CYP).

In another aspect, the recombinase is selected from the group consisting of a Cre, Cre-like, Flp, φC31, λ integrase, phage R4 recombinase, TP901-1 recombinase, a prokaryotic transposase, a eukaryotic transposase, a viral retrotransposase, a Drosophila copia-like retrotransposase and a non-viral retrotransposase. In a further embodiment, the transposase or retrotransposase is selected from the group consisting of Tn1, Tn2, Tn3, Tn4, Tn5, Tn6, Tn9, Tn10, Tn30, Tn101, Tn501, Tn903, Tn1000, Tn1681, Tn2901, Drosophila mariner, sleeping beauty transposase, Drosophila P element, maize Ac, Ds, Mp, Spm, En, dotted, Mu, I, L1, Tol2 Tc1, Tc3, Mariner (Himar 1), Mariner (mos 1) and Minos.

In one embodiment, the non-human transgenic animal substantially stops antibody diversification by gene rearrangement early in life. In a further embodiment, the non-human transgenic animal substantially stops antibody diversification within the first month of its life.

In another embodiment, the non-human transgenic animal is selected from the group consisting of rodents, rabbits, birds, cows, pigs, sheep, goats and horses. In a preferred embodiment, the rodent is a mouse or a rat.

In a certain aspect, the invention concerns a transgenic expression construct comprising a first transgene further comprising a human or humanized immunoglobulin heavy and/or light chain translocus, a self-cleaving peptide and a recombinase.

In another aspect, the invention concerns a transgenic expression construct comprising a second transgene further comprising a suicide gene that is under the control of a B-cell specific promoter, and is flanked by recombination sites recognized by a recombinase.

In one embodiment, the invention concerns a transgenic expression construct comprising a first transgene further comprising a human or humanized immunoglobulin heavy and/or light chain locus, a self-cleaving peptide and a recombinase, and, a second transgene further comprising a suicide gene that is under the control of a B-cell specific promoter, and is flanked by recombination sites recognized by the recombinase.

In one embodiment, the transgenic expression constructs described above comprise a site specific recombinase selected from the group consisting of a Cre, Cre-like, Flp, φC31, λ integrase, phage R4 and TP901-1 recombinase.

In another embodiment, the transgenic expression constructs described above comprise a recombinase that is either a prokaryotic or a eukaryotic transposase.

In yet another embodiment, the transgenic expression constructs described above comprise a recombinase that is either a viral, Drosophila copia-like or non-viral retrotransposon.

In a further embodiment, the transgenic expression constructs described above comprise a recombinase selected from the group consisting of Tn1, Tn2, Tn3, Tn4, Tn5, Tn6, Tn9, Tn10, Tn30, Tn101, Tn501, Tn903, Tn1000, Tn1681, Tn2901, Drosophila mariner, sleeping beauty transposase, Drosophila P element, maize Ac, Ds, Mp, Spm, En, dotted, Mu, I, L1, Tol2 Tc1, Tc3, Mariner (Himar 1), Mariner (mos 1) and Minos.

In a certain embodiment, the transgenic expression constructs described above comprise recombination sites that are selected from a group consisting of a lox P site, FRT site, a bacterial genomic recombination site and a phage recombination site.

In a further embodiment, the bacterial genomic recombination site is attB and the phage recombination site is an attP or a pseudo-attP or a pseudo-attB site.

In a certain embodiment, the transgenic expression constructs described above comprise a self-cleaving peptide that is obtained from viral 2A/2B or 2A-like/2B sequences.

In a further embodiment, the virus is selected from the group consisting of the picornaviridae virus family, the equine rhinitis A (ERAV) virus family, the picornavirus-like insect virus family or from the type C rotavirus family. In yet another embodiment, the virus is selected from the group consisting of the foot and mouth disease virus (FMDV), the equine rhinitis A (ERAV) virus, or the Thosea asigna virus (TaV).

In a certain embodiment, the transgenic expression constructs described above comprise a suicide gene that is specifically expressed in B-cells using a promoter/enhancer selected from the group consisting of CD19, CD20, CD21, CD22, CD23, CD24, CD40, CD72, Blimp-1, CD79b, mb-1, tyrosine kinase blk, VpreB, immunoglobulin kappa light chain, immunoglobulin lambda-light chain and immunoglobulin J-chain or modifications thereof. In a preferred embodiment, the B-cell specific promoter/enhancer is the kappa light chain gene promoter or modifications thereof.

In one aspect, this invention concerns a method for producing a non-human animal in which endogenous immunoglobulin production is suppressed through the selective expression of a suicide gene in B-cells producing endogenous immunoglobulin, while B-cells expressing human(ized) immunoglobulin, do not express the suicide gene and, therefore, propagate.

In particular, the invention concerns a non-human transgenic animal expressing the transgenic expression constructs described above.

In one embodiment, the non-human transgenic animal generates antibody diversity substantially by gene conversion.

In all aspects, preferred non-human animals include, without limitation, rodents (e.g. mice, rats), rabbits, birds (e.g. chickens, turkeys, ducks, geese, etc.), cows, pigs, sheep, goats, horses, donkeys and other farm animals. In a preferred embodiment, the non-human transgenic animal is either a mouse or a rat.

### BRIEF DESCRIPTION OF DRAWINGS

FIG. 1 is a schematic depiction of the events occurring in exogenous immunoglobulin (Ig)-expressing B-cells and endogenous immunoglobulin (Ig)-expressing B-cells. In cells with two transgenes (Ig heavy chain locus and DTA), productive rearrangement of the transgenic immunoglobulin heavy chain locus results in the expression of the heavy chain (HC) and the Cre recombinase. Subsequently, Cre-mediated recombination results in the looping-out of the DTA expression cassette. Therefore, DTA is not expressed and such exogenous B-cells, expressing the transgenic immunoglobulin HC locus, survive. Productive rearrangement of the endogenous HC locus does not result in the expression of the Cre recombinase. Therefore, the DTA expression cassette is activated and such endogenous B-cells die.

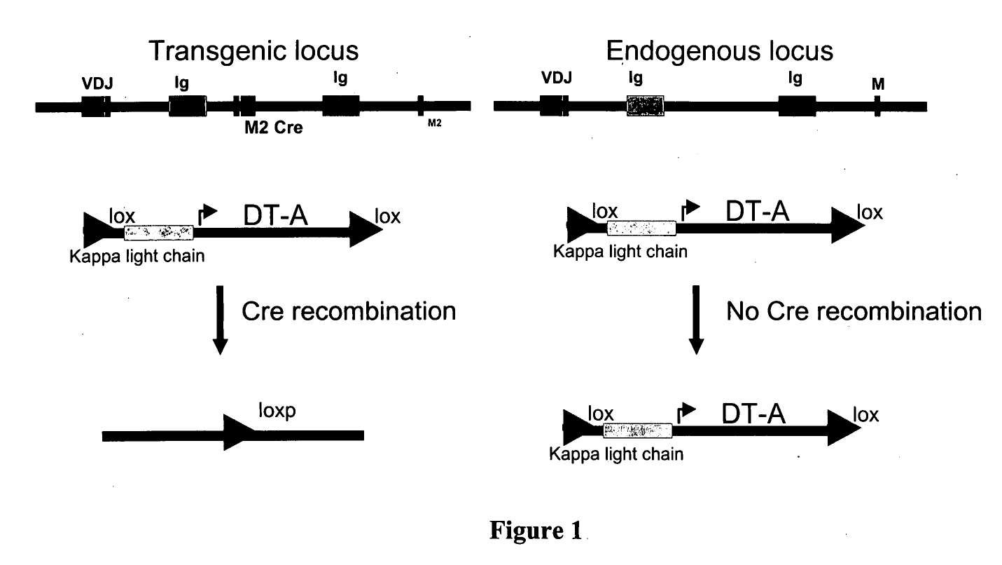

### DETAILED DESCRIPTION OF THE INVENTION

Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“B-cells” are defined as B-lineage cells that are capable of undergoing rearrangement of immunoglobulin gene segments and expressing immunoglobulin genes at some stage in their life cycle. These cells include, but are not limited to, early pro-B-cells, late pro-B-cells, large pre-B-cells, small pre-B-cells, immature B-cells, mature B-cells, memory B-cells, plasma cells, etc.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. The term “antibody” is used herein in the broadest sense and specifically covers, without limitation, monoclonal antibodies (including full length monoclonal antibodies), polyclonal antibodies, multispecific antibodies (e.g., bispecific antibodies), and antibody fragments so long as they exhibit the desired specificity.

The term “Ig gene segment” as used herein refers to segments of DNA encoding various portions of an Ig molecule, which are present in the germline of animals and humans, and which are brought together in B-cells to form rearranged Ig genes. Thus, Ig gene segments as used herein include V gene segments, D gene segments, J gene segments and C region gene segments. Functional rearrangement of VDJ or VJ segments results in expression of immunoglobulin heavy or light chain.

The term “human Ig gene translocus or locus or segment” as used herein includes both naturally occurring sequences of a human Ig gene locus or a segment thereof, degenerate forms of naturally occurring sequences of a human Ig gene locus or segments thereof, as well as synthetic sequences that encode a polypeptide sequence substantially identical to a polypeptide encoded by a naturally occurring sequence of a human Ig gene locus or a segment thereof. In this context, by “substantially” is meant that the degree of amino acid sequence identity is at least about 85%-95%, or at least about 90%-95%, or at least about 95%, or at least about 98%. In a particular embodiment, the human Ig gene segment renders the immunoglobulin molecule non-immunogenic in humans. Here, the terms “human or humanized immunoglobulin (Ig) heavy and/or light chain locus” or “human or humanized Ig locus” are used interchangeably.

The terms “human antibody” and “human immunoglobulin” are used herein to refer to antibodies and immunoglobulin molecules comprising fully human sequences.

The terms “humanized antibody” and “humanized immunoglobulin,” as used herein, mean an immunoglobulin molecule comprising at least a portion of a human immunoglobulin polypeptide sequence (or a polypeptide sequence encoded by a human immunoglobulin gene segment). The humanized immunoglobulin molecules of the present invention can be isolated from a transgenic non-human animal engineered to produce humanized immunoglobulin molecules. Such humanized immunoglobulin molecules are less immunogenic to primates, especially humans, relative to non-humanized immunoglobulin molecules prepared from the animal or prepared from cells derived from the animal. Humanized immunoglobulins or antibodies include immunoglobulins (Igs) and antibodies that are further diversified through gene conversion and somatic hypermutations in gene converting animals. Such humanized Ig or antibodies are not “human” since they are not naturally made by humans (since humans do not diversify their antibody repertoire through gene conversion) and yet, the humanized Ig or antibodies are not immunogenic to humans since they have human Ig sequences in their structure.

“Transgenes or transgene constructs” are DNA fragments with sequences encoding naturally or synthetic proteins normally not found in the animal or cells of the animal. The term “transgene construct” is used herein to refer to a polynucleotide molecule, which contains a structural “gene of interest” and other sequences facilitating gene transfer. This invention refers to two transgene constructs: (1) the human Ig locus-self-cleaving peptide-recombinase, and (2) the immune cell specific suicide transgene construct.

“A transgenic expression construct” refers to DNA fragments with sequences encoding one or several transgene constructs of the invention along with other regulatory DNA sequences needed either for temporal, or cell specific, or enhanced expression of the transgenes of interest, within specific cells of the non-human transgenic animal.

The “human(ized) Ig locus—self-cleaving peptide—recombinase transgene or transgene construct” refers to a transgene construct that is transcribed into a single mRNA, which is translated into two polypeptides, namely, the human(ized) immunoglobulin chain and the recombinase, due to a self-cleaving mechanism discussed below.

The term “self-cleaving peptide” as used herein refers to a peptide sequence that is associated with a cleavage activity that occurs between two amino acid residues within the peptide sequence itself. For example, in the 2A/2B peptide or in the 2A/2B-like peptides, cleavage occurs between the glycine residue on the 2A peptide and a proline residue on the 2B peptide. This occurs through a ‘ribosomal skip mechanism’ during translation wherein, normal peptide bond formation between the 2A glycine residue and the 2B proline residue of the 2A/2B peptide is impaired, without affecting the translation of the rest of the 2B peptide. Such ribosomal skip mechanisms are well known in the art and are known to be used by several viruses for the expression of several proteins encoded by a single messenger RNA.

The term “recombinase” as used herein refers to a group of enzymes that can facilitate site specific recombination between defined sites, called “recombination sites,” where the two recombination sites are physically separated within a single nucleic acid molecule or on separate nucleic acid molecules. The sequences of the two defined recombination sites are not necessarily identical. Within the group of recombinases there are several subfamilies including “integrases” (for example, site-specific recombinases, like Cre, Cre-like, FLP and λ integrase) and “resolvases/invertases” (for example, φC31 integrase, R4 integrase, and TP-901 integrase). The term “recombinase” also includes, but is not limited to, prokaryotic or eukaryotic transposases, viral or Drosophila copia-like or non-viral retrotransposons that include mammalian retrotransposons. Exemplary prokaryotic transposases include transposases encoded in the transposable elements of Tn1, Tn2, Tn3, Tn4, Tn5, Tn6, Tn9, Tn10, Tn30, Tn101, Tn501, Tn903, Tn1000, Tn1681, Tn2901, etc. Eukaryotic transposases include transposases encoded in the transposable elements of Drosophila mariner, sleeping beauty transposase, Drosophila P element, maize Ac and Ds elements, etc. Retrotransposases include those encoded in the elements of L1, Tol2 Tc1, Tc3, Mariner (Himar 1), Mariner (mos 1), Minos, etc. Transposases may also be selected from Mp, Spm, En, dotted, Mu, and I transposing elements.

The term “wild-type recombination site” as used herein refers to a recombination site normally used by a recombinase such as an integrase.

By “pseudo-recombination site” is meant a site at which a recombinase can facilitate recombination even though the site may not have a sequence identical to the sequence of its wild-type recombination site.

The term “suicide gene or suicide transgene” as used herein refers to a gene encoding a protein whose expression results in the death of cells expressing the gene. The protein may, for example, be a toxin (for example, diphteria toxin chain A) or an enzyme that converts a non-toxic prodrug into a toxic product (for example, thymidine kinase, carboxylesterase, carboxypeptidase, cytochrome P450 isozymes, deoxyribonucleotide kinase, nitroreductase, etc.). When the suicide gene encodes a prodrug converting enzyme, the cell expressing it dies upon exposure to the prodrug. The term “suicide gene product” as used herein refers to a protein encoded by a “suicide gene”. Suicide gene expression is driven by an immune-cell specific promoter, preferably, by a B-cell promoter.

The term “sequences that enable inactivation of suicide gene expression” refers to the recombination sites flanking the suicide gene, that are recognized by a recombinase.

The term “prodrug” means a compound that is convertible in vivo metabolically into a toxic product, metabolite or drug.

The terms “endogenous Ig (immunoglobulin)-expressing B-cells” and “endogenous B-cells” are used interchangeably, and refer to those B-cells that express the animal's endogenous immunoglobulin locus. B-cells of the invention contain a suicide gene in their genome. Endogenous B-cells express the suicide gene and, as a result, are eventually depleted and the animal's endogenous Ig expression is therefore suppressed.

The terms “exogenous Ig (immunoglobulin)-expressing B-cells” and “exogenous B cells” refer to those B-cells of a non-human animal that undergo productive rearrangement of an exogenous human(ized) Ig translocus introduced into such B-cells. The human(ized) Ig locus is introduced into such B-cells as part of an expression construct, also encoding a site-specific recombinase. Productive rearrangement of the human(ized) Ig locus results in the expression of the human(ized) Ig and the transgene encoded-recombinase. As a result, the suicide gene is excised from the genome of the B-cells and the cells escape cell death. Thus preferably, the Ig product expressed by the transgenic animal is the human(ized) immunoglobulin.

By “selective expression of the suicide gene” is meant, expression of the suicide gene product preferably within immune cells, more preferably within B-cells, and most preferably, within endogenous B-cells. Selective expression of the suicide gene within immune cells or B-cells is achieved by using an immune-specific or a B-cell specific promoter respectively, to drive suicide gene expression.

The term “selective inactivation” refers to the selective excision of the suicide gene, or parts thereof, from the genome of exogenous B-cells. Since the suicide gene is flanked by recombination sites recognized by the transgene encoded recombinase expressed in exogenous B-cells, the suicide gene is excised out or inactivated.

“Depletion” of Ig producing cells is defined as the partial or complete killing, dying and/or removal of endogenous B-cell populations expressing non-human or non-humanized Ig. Depletion of endogenous B-cells may be further effective when the transgenic animal of choice is one wherein antibody rearrangement stops early in life, as explained further below.

“Selective suppression of endogenous immunoglobulin production” refers to selective suppression of the production of endogenous immunoglobulin of the non-human transgenic animal, due to the depletion of endogenous B-cells expressing the suicide gene. Thus, the immunoglobulin product predominantly expressed by the transgenic animal is the human(ized) immunoglobulin.

The terms “antibody diversity” and “antibody repertoire” are used interchangeably, and refer to the total of all antibody specificities that an organism is capable of expressing.

An Ig locus having the capacity to undergo gene rearrangement and gene conversion is also referred to herein as a “functional” Ig locus, and the antibodies with a diversity generated by a functional Ig locus are also referred to herein as “functional” antibodies or a “functional” repertoire of antibodies.

The term “monoclonal antibody” is used to refer to an antibody molecule synthesized by a single clone of B-cells.

The term “polyclonal antibody” is used to refer to a population of antibody molecules synthesized by a population of B-cells.

The terms “polynucleotide” and “nucleic acid” are used interchangeably, and, when used in singular or plural, generally refer to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “non-human (transgenic) animal” as used herein includes, but is not limited to, mammals such as, for example, non-human primates, rodents (e.g. mice and rats), non-rodent mammals, such as, for example, rabbits, pigs, sheep, goats, cows, pigs, horses and donkeys, and birds (e.g., chickens, turkeys, ducks, geese and the like). The term “non-primate animal” as used herein includes, but is not limited to, mammals other than primates, including but not limited to the mammals specifically listed above.

The phrase “animals which create antibody diversity substantially by gene conversion and/or somatic hypermutation to create primary antibody repertoires” or “gene converting animals” and their grammatical equivalents, are used to refer to such animals in which the predominant mechanism of antibody diversification is gene conversion and/or hypermutation as opposed to gene rearrangement. Such animals include, but are not limited to, rabbits, birds (e.g., chickens, turkeys, ducks, geese and the like), cows and pigs. Particularly preferred non-human animals are rabbits and chickens.

By animals “stopping antibody gene rearrangement early in life” is meant those animals where the rearrangement of immunoglobulin genes stops typically within the first month of life. Examples of such animals are, without limitation, rabbits, birds (e.g. chickens), sheep, goats, cattle, swine and horses.

### DETAILED DESCRIPTION

The present invention provides methods for the suppression of the endogenous immunoglobulin production in non-human animals, for example with the aim to render the animals more suitable for the expression of human(ized) immunoglobulin(s).

According to the present invention, endogenous immunoglobulin production is selectively suppressed in non-human transgenic animals expressing exogenous immunoglobulin sequences, like human(ized) immunoglobulin(s), through the selective expression of a suicide gene in B-cells expressing endogenous immunoglobulin. The suicide gene is integrated into the animal's genome as a transgene, and may, for example, be introduced as part of a transgenic expression construct that also introduces the human(ized) Ig translocus or separately, e.g. using a separate transgenic expression construct. In the latter case, the two expression constructs may be introduced simultaneously or at different times into the transgenic animal.

The suicide gene is expressed in B-cells of the animal by means of an immune-specific promoter, preferably a B-cell specific promoter, and is flanked by recombination sequences recognized by a recombinase. Accordingly, the suicide gene, flanked by recombination sequences, will initially be present in all B cells of the animal. In “exogenouse B-cells” productive rearrangement of an exogenous immunoglobulin translocus encoding a human(ized) immunoglobulin—self cleaving peptide—recombinase molecule results in the selective expression of the recombinase in such B-cells. The recombinase recognizes the recombination sites flanking the suicide gene in such cells. As a result, the suicide gene, is excised out selectively in exogenous B-cells which, consequently, escape cell death. In contrast, productive rearrangment of an endogenous immunoglobulin locus in endogenous B-cells does not result in expression of the recombinase. Consequently, suicide gene expression in endogenous B-cells results in death of this cell population, and consequently, endogenous immunoglobulin production is suppressed without suppressing the expression of human(ized) immunoglobulin by the non-human transgenic animal.

Transgenes are DNA fragments with sequences encoding for one or several naturally or synthetic proteins not normally found in the animal or cells of the animal. The DNA fragment(s) may be introduced into the animal's genome by a variety of techniques including microinjection of pronuclei, transfection, nuclear transfer cloning, sperm-mediated gene transfer, testis-mediated gene transfer, and the like. The present invention refers to two transgenes or transgene constructs, (1) the human Ig locus—self-cleaving peptide—recombinase transgene, and (2) the immune cell specific suicide transgene. Each transgene is operatively liked to its own regulatory sequences. For example, expression of the suicide transgene may be driven by a B-cell specific promoter. The two transgene constructs may be present on two separate vector(s) or on the same vector (plasmid). In one embodiment, the two transgene constructs may be introduced at separate times. Alternatively, both transgene constructs may be introduced simultaneously into the animal. In a preferred embodiment, the expression of the suicide transgene is timed to occur after heavy chain rearrangement has taken place. As will be apparent from the mechanisms discussed below, this allows time for the expression of the recombinase in the exogenous B-cells, and accordingly, time for recombinase-mediated excision of the suicide gene from the genome of exogenous B cells, thus shutting down suicide gene expression in such cells. Additionally, the vectors used in the methods of the present invention may contain DNA sequences that code for antibiotic selection markers like gentamycin, neomycin kanamycin etc. to enable selection.

In one aspect of the present invention, the transgene comprises DNA sequences encoding for a self cleaving peptide (for example, 2A peptide or 2A-like peptide). Insertion of a self-cleaving peptide-encoding sequence between the immunoglobulin-encoding sequence and a recombinase-encoding sequence in the transgene results in production of one messenger RNA. Translation of this mRNA, however, results in two separate proteins, the immunoglobulin(s) and the recombinase, due to the peptide's self-cleaving mechanism. Therefore, expression of the recombinase can be coupled to the functional rearrangement of VDJ or VJ segments.

In one such embodiment of the invention, the self-cleaving is mediated by 2A/2B peptides, or 2A-like/2B sequences of viruses that include the picornaviridae virus family, the equine rhinitis A (ERAV) virus family, the picornavirus-like insect virus family or from the type C rotavirus family. The picornaviridae virus family includes the entero-, rhino-, cardio- and aphtho- and foot-and-mouth disease (FMDV) viruses. The picornavirus-like insect virus family includes viruses such as the infectious flacherie virus (IFV), the Drosophila C virus (DCV), the acute bee paralysis virus (ABPV) and the cricket paralysis virus (CrPV) and the insect virus Thosea asigna virus (TaV). The type C rotavirus family includes the bovine, porcine and human type C rotaviruses. In further embodiments, the cleavage sequences may include 2A-like/2B sequences from either the poliovirus, rhinovirus, coxsackie virus, encephalomyocarditis virus (EMCV), mengovirus, the porcine teschovirus-1, or the Theiler's murine encephalitis virus (TMEV), etc. In a preferred embodiment, the self-cleaving protein sequence is either the 2A/2B peptide of the foot and mouth disease virus (FMDV), the equine rhinitis A (ERAV) virus, or the Thosea asigna virus (TaV); Palmenberg et al., Virology 190:754-762 (1992), Ryan et al., J Gen Virol 72:2727-2732 (1991), Donnelly et al., J Gen Virol 82:1027-1041 (2001), Donnelly et al., J Gen Virol 82:1013-1025 (2001), Szymaczak et al., Nature Biotech 22(5):589-594 (2004).

The other transgene used in the methods of the present invention encodes for a site-specific recombinase. Site-specific recombinases catalyze homologous recombination between two nucleic acids, e.g. DNA segments. These recombinases recognize very specific sequences in both partners of the recombination. While the mechanism of catalysis might be different for different types of site-specific recombinases, they are all included herein, regardless of the underlying mechanism, and are suitable for the practice of the present invention.

In a particular embodiment, the recombinase may, for example, be a Cre, Flp recombinase, or the like. Cre and Flp are the two most commonly used enzymes, which only act on very specific DNA sequences. Cre catalyzes the recombination of DNA between two 34-base pair long loxP sites, while Flp targets the frt site. The use of Cre recombinase for site-specific recombination of DNA in eukaryotic cells is described in U.S. Pat. No. 4,959,317. The use of site specific recombinase for the transfection of eukaryotic cells is described in U.S. Pat. No. 6,632,672. Site specific recombination in general is described in U.S. Pat. No. 4,673,640. Cre/loxP based cloning systems are commercially available, for example, from BD Biosciences-Clontech, Palo Alto, Calif. (Creator™), or Invitrogen, Carlsbad, Calif. (Echo™).

In another embodiment, the recombinase may be a site-specific recombinase encoded by a phage selected from the group consisting of λ integrase, φC31, TP901-1, and R4. φC31 and R4 belong in the integrase family of site-specific recombinases, while TP901-1 belongs to the family of extended resolvases. The R4 integrase is a site-specific, unidirectional recombinase derived from the genome of phage R4 of Streptomyces parvulus. The site-specific integrase TP901-1 is encoded by phage TP901-1 of Lactococcus lactis subsp. cremoris. λ is a temperate bacteriophage that infects E. coli. The phage has one attachment site for recombination (attP) and the E. coli bacterial genome has an attachment site for recombination (attB). In the context of the present invention, wild-type recombination sites can be derived, for example, from the homologous system and associated with heterologous sequences. Thus, the attB site can be placed in other systems to act as a substrate for an integrase. In yet another embodiment, the recombinase may catalyze recombination between a bacterial genomic recombination site (attB) and a phage genomic recombination site (attP), or the first site may comprises a pseudo-attB site and/or the second site may comprises a pseudo-attP site, or vice versa (Groth et al., Proc. Nat. Acad. Sci., 2000, 97: 5995-6000; Olivares et al., Nature Biotechnol. 2002, 20(11): 1124-8); (Thyagarajan et al., Mol. and Cell. Biol., 2001, 21: 3926-3934); Hollis et al., Repro. Biol. and Endocrinol., 2003, 1:79. By “pseudo-recombination site” is meant a site at which recombinase can facilitate recombination even though the site may not have a sequence identical to the sequence of its wild-type recombination site.

In yet another embodiment, the recombinase can be a transposase or a retrotransposase. Transposons or retrotransposases, are enzymes that catalyze their transposition by a cut and paste mechanism and can be used for the transfer or insertion of any transgene. They provide non-viral and non-homologous methods for the insertion or transfer of any DNA sequence into the genomes of a wide range of species, including vertebrates like humans, bird, rodents, etc. For example, the Drosophila element mariner was shown to transpose itself into chicken germ lines, Sherman et al., Nature Biotechnol., 16:1050-1053 (1998). Long term transgene expression or efficient insertion of transposon DNA, using the sleeping beauty transposase system into mammalian systems like the mouse and human genomes have been demonstrated by Yant et al., Nature Genetics, 25: 35-41 (2000); Dupuy et al., Proc. Nat. Acad. Sci., 99: 4495-4499 (2002) and Geurts et al., Mol. Therapy, 8: 108-117 (2003). Other transposes like L1, Tol2 Tc1, Tc3, Mariner (Himar 1), Mariner (mos 1), Minos have been shown to be active in vertebrate species and are thus useful for gene transfer or as insertional mutagenesis vectors, Largaespada, David A., Repro. Biol. and Endocrinol., 1:80 (2003). Exemplary transposases include, but are not limited to, prokaryotic or eukaryotic transposases, viral, Drosophila copia-like or non-viral retrotransposons which include mammalian retrotransposons, etc. Prokaryotic transposases include transposases encoded in the transposable elements of Tn1, Tn2, Tn3, Tn4, Tn5, Tn6, Tn9, Tn10, Tn30, Tn101, Tn501, Tn903, Tn1000, Tn1681, Tn2901, etc. Eukaryotic transposases include transposases encoded in the transposable elements of Drosophila mariner, sleeping beauty transposase, Drosophila P element, maize Ac and Ds elements, etc. Retrotransposases include those encoded in the elements of L1, Tol2 Tc1, Tc3, Mariner (Himar 1), Mariner (mos 1), Minos, etc. Transposases may also be selected from Mp, Spm, En, dotted, Mu, and I transposing elements.

In one aspect of the invention, suicide genes flanked by the site-specific recombinase recognition sites described above, are used to kill the endogenous B-cells selectively. Flanking the suicide gene with recombination sites results in inactivation of the suicide gene upon expression of the recombinase or transposon in exogenous B-cells only. Inactivation of suicide gene expression may be accomplished through excision of the suicide gene or parts thereof. Alternatively, sequences necessary for the expression of the suicide gene may be targeted for excision. According to another approach, suicide gene expression may be inactivated by the inversion or insertion of DNA fragments, due to transposon jumping, transposon insertion or inversion.

The suicide genes used in the practice of the invention include toxin genes and non-toxic, prodrug converting enzymes which yield toxic products. Active toxins and fragments thereof which can be used in the methods of the invention include, for example, bacterial toxins or fragments thereof like diphtheria toxin chain A (DTA), Shiga toxin, exotoxin A chain (from Pseudomonas aeruginosa), etc., or plant or fungal toxins and their nonbinding active fragments like ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes, insecticidal toxins, reptilian venoms, etc. In a preferred embodiment, the toxin used is the diphtheria toxin chain A (DTA).

When the suicide gene that is introduced into the target B-cell encodes a prodrug converting enzyme, the enzyme activates a specific non-toxic prodrug to create toxic metabolites that eventually kill the target B-cell. In this approach, a two-step treatment method may be designed to suppress endogenous B-cell production. In the first step, the gene for a foreign enzyme may be expressed or delivered to the B-cell in a variety of ways known in the art. In the second step, a prodrug that is administered to the animal is activated to a toxic metabolite by the B-cell expressing the enzyme eventually killing it.

Prodrug converting enzymes useful as suicide genes are generally found in two major classes. The first class are enzymes of non-mammalian origin, with or without human counterparts. Examples include viral thymidine kinase (TK), bacterial cytosine deaminase (CD), bacterial carboxypeptidase G2 (CPG2), purine nucleotide phosphorylase (PNP), thymidine phosphorylase (TP), nitroreductase (NR), D-amino acid oxidase (DAAO), xanthine-guanine phosphoribosyl transferease (XGPRT), penicillin-G amidase (PGA), β-lactamase, multiple drug activation enzyme (MDAE), β-galactosidase (β-Gal), horseradish peroxidase (HRP) and deoxyribonucleotide kinase (DRNK). The second class consist of enzymes of human origin. These include deoxycytidine kinase (dCK), carboxyesterases (CEs), carboxypeptidase A (CPA), β-glucuronidase (-Glu), and cytochrome P450 (CYP) isozymes. Additional examples of enzyme-prodrug systems are listed in Table 1 of Methods in Molecular Medicine: Suicide Gene Therapy, Methods and Reviews, edited by Caroline J Springer, Humana Press, 2004, which are hereby incorporated by reference. Thus, prodrug converting enzymes used as suicide genes in this invention include, but are not limited to, enzymes of non-mammalian, non-human origin and human origin, as described above.

Suitable prodrugs that are useful in this invention include, but are not limited to, ganciclovir, aciclovir, 5-(aziridin-1-yl)-2,3 dinitrobenamide, capecitabine, irinotecan, carbamate-based 20(S)-camptothecins, dinitrobenzamide aziridine CB 1954 and its nitrogen mustard analogue SN 23862, 2-aminoanthracene (2-AA) and 4-ipomeanol (4-IM), etc. Additional examples of prodrugs that are useful in this invention with their corresponding activating enzymes are listed in Table 1 of Methods in Molecular Medicine: Suicide Gene Therapy, Methods and Reviews, edited by Caroline J Springer, Humana Press, 2004, which is hereby incorporated by reference. Enzyme-activated prodrugs may sometimes be used in combination with other cell-killing methods, for example, with radiosensitizing drugs like etanidazole, fluosol, misonidazole, nimorazole, temoporfin, tirapazamine, or with the expression of other apoptotic agents like caspases, leading to synergistic effects that kill target endogenous Ig-expressing B-cells.

Thus, the expression of a suicide gene may eliminate B-cells expressing endogenous immunoglobulin by a variety of mechanisms. For example, expression of DTA results in the inhibition of protein synthesis and subsequent cell death. Thymidine kinase phosphorylates ganciclovir and aciclovir to their corresponding monophosphate forms, which are subsequently converted to toxic triphosphate derivatives by cellular kinases. Incorporation of the toxic triphosphates into the DNA of dividing cells results in cell death. Nitroreductase converts 5-(aziridin-1-yl)-2,3 dinitrobenamide, into 2- and 4-hydroxylamino derivatives, whereupon the non-enzymatic reaction of the 4-hydroxylamino derivative with cellular thio-esters generates a potent cytotoxic bifunctional alkylating agent capable of cross-linking DNA. Suicide genes known in the art are useful in this invention without being bound or limited by the mechanism(s) in which these suicide genes act to eliminate the endogenous B-cells.

In a preferred embodiment of the invention, the suicide gene encodes diphteria toxin chain A (DTA) flanked by wild-type or pseudo-recombination sites (for example, lox P sites recognized by Cre or FRT sites recognized by Flp).

In another embodiment of the invention, the suicide gene encodes thymidine kinase flanked by wild-type- or pseudo recombination sites.

In one embodiment, the suicide gene, flanked by site-specific recombinase recognition sites, is introduced into an animal on a separate transgenic vector, either before, concurrently, or after the introduction of the human Ig locus—self-cleaving peptide—recombinase transgene. In another embodiment, the suicide gene, flanked by site-specific recombinase recognition sites, is introduced on the same transgenic vector as the one for the human Ig locus-recombinase transgene into an animal. In all aspects, the suicide gene integrates into the animal's genome and it's expression is driven by an immune-cell specific promoter ensuring it's expression specifically in immune cells alone and preferably, by a B-cell specific promoter ensuring it's expression specifically in B-cells alone, and not in other cell types.

Expression of the suicide gene is controlled by a B-cell specific promoter so that its expression is ‘switched off’ in non-B-cells or tissues of the non-human transgenic animal. Promoters (and enhancers), or variants or engineered portions thereof, that control the expression of B-cell specific genes, are useful for such B-cell specific expression of the suicide gene. Examples of promoters/enhancers of B-cell specific genes include, but are not limited to, promoters/enhancers of CD 19, CD20, CD21, CD22, CD23, CD24, CD40, CD72, Blimp-1, CD79b (also known as B29 or Ig beta), mb-I (also known as Ig alpha), tyrosine kinase blk, VpreB, immunoglobulin kappa light chain, immunoglobulin lambda-light chain, immunoglobulin J-chain, etc. In a preferred embodiment, the kappa light chain promoter/enhancer drives the B-cell specific expression of the suicide gene.

Thus, suppression of the endogenous immunoglobulin production results in the dominant expression of the human(ized) Ig translocus. In other words, depletion of endogenous B-cells leads to enrichment of human(ized) antibodies. Preferably, the enrichment of exogenous B-cells is close to 100%.

In yet another aspect of the invention, the transgene encodes immunoglobulin heavy chains and/or immunoglobulin light chains or parts thereof. The loci can be in germline configuration or in a rearranged form. The coding sequences or parts thereof may code for human immunoglobulins resulting in the expression of human(ized) antibodies.

The transgene(s) encoding human(ized) antibodies contain(s) an Ig locus or a large portion of an Ig locus, containing one or several human Ig segments (e.g., a human Ig V, D, J or C gene segment). Alternatively, the transgene is a human immunoglobulin locus or a large portion thereof. The transgene containing such a human Ig locus or such modified Ig locus or modified portion of an Ig locus, also referred to herein as “a human(ized) Ig translocus”, is capable of undergoing gene rearrangement in the transgenic non-human animal thereby producing a diversified repertoire of antibodies having at least a portion of a human immunoglobulin polypeptide sequence.

Immunoglobulin heavy and light chain genes comprise several segments encoded by individual genes and separated by intron sequences. Thus genes for the human immunoglobulin heavy chain are found on chromosome 14. The variable region of the heavy chain (VH) comprises three gene segments: V, D and J segments, followed by multiple genes coding for the C region. The V region is separated from the C region by a large spacer, and the individual genes encoding the V, D and J segments are also separated by spacers.

There are two types of immunoglobulin light chains: κ and λ. Genes for the human κ light chain are found on chromosome 2 and genes for the human λ light chain are found on chromosome 22. The variable region of antibody light chains includes a V segment and a J segment, encoded by separate gene segments. In the germline configuration of the κ light chain gene, there are approximately 100-200 V region genes in linear arrangement, each gene having its own leader sequence, followed by approximately 5 J gene segments, and C region gene segment. All V regions are separated by introns, and there are introns separating the V, J and C region gene segments as well.

The immune system's capacity to protect against infection rests in a genetic machinery specialized to create a diverse repertoire of antibodies. Antibody-coding genes in B-cells are assembled in a manner that allows to countless combinations of binding sites in the variable (V) region. It is estimated that more than 10¹² possible binding structures arise from such mechanisms. In all animals, including humans, the antibody-making process begins by recombining variable (V), diversity (D) and joining (J) segments of the immunoglobulin (Ig) locus. Following this step, depending on the animal species, two general mechanisms are used to produce the diverse binding structures of antibodies.

In some animals, such as human and mouse, there are multiple copies of V, D and J gene segments on the immunoglobulin heavy chain locus, and multiple copies of V and J gene segments on the immunoglobulin light chain locus. Antibody diversity in these animals is generated primarily by gene rearrangement, i.e., different combinations of gene segments to form rearranged heavy chain variable region and light chain variable region. In other animals (e.g., rabbit, birds, e.g., chicken, goose, and duck, sheep, goat, and cow), however, gene rearrangement plays a smaller role in the generation of antibody diversity. For example, in rabbit, only a very limited number of the V gene segments, most often the V gene segments at the 3′ end of the V-region, is used in gene rearrangement to form a contiguous VDJ segment. In chicken, only one V gene segment (the one adjacent to the D region, or “the 3′ proximal V gene segment”), one D segment and one J segment are used in the heavy chain rearrangement; and only one V gene segment (the 3′ proximal V segment) and one J segment are used in the light chain rearrangement. Thus, in these animals, there is little diversity among initially rearranged variable region sequences resulting from junctional diversification. Further diversification of the rearranged Ig genes is achieved by gene conversion, a process in which short sequences derived from the upstream V gene segments replace short sequences within the V gene segment in the rearranged Ig gene. Additional diversification of antibody sequences may be generated by hypermutation.

Immunoglobulins (antibodies) belong into five classes (IgG, IgM, IgA, IgE, and IgD, each with different biological roles in immune defense. The most abundant in the blood and potent in response to infection is the IgG class. Within the human IgG class, there are four sub-classes (IgG1, IgG2, IgG3 and IgG4 isotypes) determined by the structure of the heavy chain constant regions that comprise the Fc domain. The F(ab) domains of antibodies bind to specific sequences (epitopes) on antigens, while the Fc domain of antibodies recruits and activates other components of the immune system in order to eliminate the antigens.

Native antibodies and immunoglobulins are usually heterotetrameric glycoproteins of about 150,000 daltons, composed of two identical light (L) chains and two identical heavy (H) chains. Each light chain is linked to a heavy chain by covalent disulfide bond(s), while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al., J. Mol. Biol. 186:651 (1985); Novotny and Haber, Proc. Natl. Acad. Sci. U.S.A. 82:4592 (1985)).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, connected by three CDRs. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

The creation of human-animal translocus allows for the creation of transgenic animals that express diversified, high-affinity human(ized) (polyclonal) antibodies in high yields. In general, the humanization of an immunoglobulin (Ig) locus in a non-human animal involves the integration of one or more human Ig gene segments into the animal's genome to create human(ized) immunoglobulin loci. Thus, creation of a human(ized) Ig heavy chain locus involves the integration of one or more V and/or D and/or J segments, and/or C region segments into the animal's genome. Similarly, the creation of a humanized Ig light chain locus involves the integration of one or more V and/or J segments, and/or C region segments into the animal's genome.

Regardless of the chromosomal location, the human(ized) Ig locus of the present invention has the capacity to undergo gene rearrangement and gene conversion and hypermutation in the non-human animal, thereby producing a diversified repertoire of human(ized) Ig molecules. An Ig locus having the capacity to undergo gene rearrangement and gene conversion is also referred to as a “functional” Ig locus and the antibodies with a diversity generated by a functional Ig locus are also referred to as “functional” antibodies or a “functional” repertoire of antibody molecules.

In one aspect, animals in which diversification of the antibody repertoire stops early in life are useful in the current invention. B-cells develop from hematopoietic stem cells. Prior to antigen exposure, B-cells undergo a series of maturation steps the end product of which is a mature B-cell, which expresses a unique membrane-associated IgM and often IgD on its cell surface along with other cell surface signaling molecules. While in humans, antibody diversification by gene rearrangement occurs throughout life, in other animals the diversification of antibody repertoire stops early in life, typically within the first month of life.

In animals where rearrangement of immunoglobulin genes stops early in life, killing all or most of B-cells produced during this limited period of time effectively results in lasting or permanent arrest of endogenous immunoglobulin production. In transgenic animals, which contain one or several human or humanized immunoglobulin transloci, this enables the production of human or humanized immunoglobulin, in the absence of endogenous immunoglobulin production of the animal. In this way, the expression of the endogenous immunoglobulin(s) can be effectively suppressed in animals where gene rearrangement stops early in live. Examples of such animals are, without limitation, rabbits, birds (e.g. chickens), sheep, goats, cattle, swine and horses.

According to the present invention, a transgenic animal capable of making human(ized) immunoglobulins is made, by introducing into a recipient cell or cells of an animal, one or more of the transgenic vectors described herein above, one of which carries a human(ized) Ig locus, and deriving an animal from the genetically modified recipient cell or cells.

The recipient cells may, for example, be from non-human animals which generate antibody diversity by gene conversion and/or hypermutation, e.g., bird (such as chicken), rabbit, cows and the like. In such animals, the 3′proximal V gene segment is preferentially used for the production of immunoglobulins. Integration of a human V gene segment into the Ig locus on the transgene vector, either by replacing the 3′proximal V gene segment of the animal or by being placed in close proximity of the 3′proximal V gene segment, results in expression of human V region polypeptide sequences in the majority of immunoglobulins. Alternatively, a rearranged human V(D)J segment may be inserted into the J locus of the immunoglobulin locus on the transgene vector.

The transgenic vectors containing the genes of interest containing the human(ized) Ig locus and the suicide gene may be introduced into the recipient cell or cells and then integrated into the genome of the recipient cell or cells by random integration or by targeted integration.

For random integration, a transgenic vector containing a human(ized) Ig locus can be introduced into an animal recipient cell by standard transgenic technology. For example, a transgenic vector can be directly injected into the pronucleus of a fertilized oocyte. A transgenic vector can also be introduced by co-incubation of sperm with the transgenic vector before fertilization of the oocyte. Transgenic animals can be developed from fertilized oocytes. Another way to introduce a transgenic vector is by transfecting embryonic stem cells and subsequently injecting the genetically modified embryonic stem cells into developing embryos. Alternatively, a transgenic vector (naked or in combination with facilitating reagents) can be directly injected into a developing embryo. Ultimately, chimeric transgenic animals are produced from the embryos which contain the human(ized) Ig transgene integrated in the genome of at least some somatic cells of the transgenic animal.

In a particular embodiment, a transgene containing a human(ized) Ig locus is randomly integrated into the genome of recipient cells (such as fertilized oocyte or developing embryos) derived from animal strains with an impaired expression of endogenous immunoglobulin genes. The use of such animal strains permits preferential expression of immunoglobulin molecules from the human(ized) transgenic Ig locus. Examples for such animals include the Alicia and Basilea rabbit strains, as well as agammaglobinemic chicken strain, as well as immunoglobulin knock-out mice. Alternatively, transgenic animals with human(ized) immunoglobulin transgenes or loci can be mated with animal strains with impaired expression of endogenous immunoglobulins. Offspring homozygous for an impaired endogenous Ig locus and a human(ized) transgenic Ig locus can be obtained.

For targeted integration, a transgenic vector can be introduced into appropriate animal recipient cells such as embryonic stem cells or already differentiated somatic cells. Afterwards, cells in which the transgene has integrated into the animal genome and has replaced the corresponding endogenous Ig locus by homologous recombination can be selected by standard methods See for example, Kuroiwa et al, Nature Genetics 2004, Jun. 6. The selected cells may then be fused with enucleated nuclear transfer unit cells, e.g. oocytes or embryonic stem cells, cells which are totipotent and capable of forming a functional neonate. Fusion is performed in accordance with conventional techniques which are well established. Enucleation of oocytes and nuclear transfer can also be performed by microsurgery using injection pipettes. (See, for example, Wakayama et al., Nature (1998) 394:369.) The resulting egg cells are then cultivated in an appropriate medium, and transferred into synchronized recipients for generating transgenic animals. Alternatively, the selected genetically modified cells can be injected into developing embryos which are subsequently developed into chimeric animals.

Further, according to the present invention, a transgenic animal capable of producing human(ized) immunoglobulins can also be made by introducing into a recipient cell or cells, one or more of the recombination vectors described herein above, one of which carries a human Ig gene segment, linked to 5′ and 3′ flanking sequences that are homologous to the flanking sequences of the endogenous Ig gene segment, then selecting cells in which the endogenous Ig gene segment is replaced by the human Ig gene segment by homologous recombination, and deriving an animal from the selected genetically modified recipient cell or cells.

Similar to the target insertion of a transgenic vector, cells appropriate for use as recipient cells in this approach include embryonic stem cells or already differentiated somatic cells. A recombination vector carrying a human Ig gene segment can be introduced into such recipient cells by any feasible means, e.g., transfection. Afterwards, cells in which the human Ig gene segment has replaced the corresponding endogenous Ig gene segment by homologous recombination, can be selected by standard methods. These genetically modified cells can serve as nuclei donor cells in a nuclear transfer procedure for cloning a transgenic animal. Alternatively, the selected genetically modified embryonic stem cells can be injected into developing embryos which can be subsequently developed into chimeric animals.

In a specific embodiment, the transgene constructs of the invention may be introduced into the transgenic animals during embryonic life by directly injecting the transgenes into the embryo or indirectly by injecting them into the pregnant mother or into the egg-laying hen. As a consequence, the endogenous B-cells expressing the animal's immunoglobulin molecules may be depleted and hence transgenic offspring will predominantly produce human(ized) antibodies in response to immunization with antigens.

Transgenic animals produced by any of the foregoing methods form another embodiment of the present invention. The transgenic animals have at least one, i.e., one or more, human(ized) Ig loci in the genome, from which a functional repertoire of human(ized) antibodies is produced and a suicide gene.

In a specific embodiment, the present invention provides transgenic rabbits having one or more human(ized) Ig loci and a suicide gene in the genome. The transgenic rabbits of the present invention are capable of rearranging and gene converting the human(ized) Ig loci, and expressing a functional repertoire of human(ized) antibodies.

In another specific embodiment, the present invention provides transgenic chickens having one or more human(ized) Ig loci and a suicide gene in the genome. The transgenic chickens of the present invention are capable of rearranging and gene converting the human(ized) Ig loci, and expressing a functional repertoire of human(ized) antibodies. In another specific embodiment, the present invention provides transgenic mice with one or more human(ized) V regions and a suicide gene in the genome. The human(ized) V region comprises at least two human V gene segments flanked by non-human spacer sequences. The transgenic mice are capable of rearranging the human V elements and expressing a functional repertoire of antibodies.

Immunization with antigen leads to the production of human(ized) antibodies against the same antigen in said transgenic animals.

Although preferred embodiments of the present invention are directed to transgenic animals having human(ized) Ig loci and at least one suicide gene to deplete endogenous B-cells and producing human(ized) polyclonal antisera, it is to be understood that transgenic animals having primatized Ig loci and primatized polyclonal antisera are also within the spirit of the present invention. Similar to human(ized) polyclonal antisera compositions, primatized polyclonal antisera compositions are likely to have a reduced immunogenicity in human individuals.

Once a transgenic non-human animal capable of producing diversified human(ized) immunoglobulin molecules is made (as further set forth below), human(ized) immunoglobulins and human(ized) antibody preparations against an antigen can be readily obtained by immunizing the animal with the antigen. A variety of antigens can be used to immunize a transgenic host animal. Such antigens include, microorganism, e.g. viruses and unicellular organisms (such as bacteria and fungi), alive, attenuated or dead, fragments of the microorganisms, or antigenic molecules isolated from the microorganisms.

Preferred bacterial antigens for use in immunizing an animal include purified antigens from Staphylococcus aureus such as capsular polysaccharides type 5 and 8, recombinant versions of virulence factors such as alpha-toxin, adhesin binding proteins, collagen binding proteins, and fibronectin binding proteins. Preferred bacterial antigens also include an attenuated version of S. aureus, Pseudomonas aeruginosa, enterococcus, enterobacter, and Klebsiella pneumoniae, or culture supernatant from these bacteria cells. Other bacterial antigens which can be used in immunization include purified lipopolysaccharide (LPS), capsular antigens, capsular polysaccharides and/or recombinant versions of the outer membrane proteins, fibronectin binding proteins, endotoxin, and exotoxin from Pseudomonas aeruginosa, enterococcus, enterobacter, and Klebsiella pneumoniae.

Preferred antigens for the generation of antibodies against fingi include attenuated version of fungi or outer membrane proteins thereof, which fungi include, but are not limited to, Candida albicans, Candida parapsilosis, Candida tropicalis, and Cryptococcus neoformans.

Preferred antigens for use in immunization in order to generate antibodies against viruses include the envelop proteins and attenuated versions of viruses which include, but are not limited to respiratory synctial virus (RSV) (particularly the F-Protein), Hepatitis C virus (HCV), Hepatits B virus (HBV), cytomegalovirus (CMV), EBV, and HSV.

Therapeutic antibodies can be generated for the treatment of cancer by immunizing transgenic animals with isolated tumor cells or tumor cell lines; tumor-associated antigens which include, but are not limited to, Her-2-neu antigen (antibodies against which are useful for the treatment of breast cancer); CD 19, CD20, CD22 and CD53 antigens (antibodies against which are useful for the treatment of B-cell lymphomas), (3) prostate specific membrane antigen (PMSA) (antibodies against which are useful for the treatment of prostate cancer), and 17-1A molecule (antibodies against which are useful for the treatment of colon cancer).

The antigens can be administered to a transgenic host animal in any convenient manner, with or without an adjuvant, and can be administered in accordance with a predetermined schedule.

After immunization, serum or milk from the immunized transgenic animals can be fractionated for the purification of pharmaceutical grade polyclonal antibodies specific for the antigen. In the case of transgenic birds, antibodies can also be made by fractionating egg yolks. A concentrated, purified immunoglobulin fraction may be obtained by chromatography (affinity, ionic exchange, gel filtration, etc.), selective precipitation with salts such as ammonium sulfate, organic solvents such as ethanol, or polymers such as polyethyleneglycol.

The fractionated human(ized) antibodies may be dissolved or diluted in non-toxic, non-pyrogenic media suitable for intravenous administration in humans, for instance, sterile buffered saline.

The antibody preparations used for administration are generally characterized by having immunoglobulin concentrations from 0.1 to 100 mg/ml, more usually from 1 to 10 mg/ml. The antibody preparation may contain immunoglobulins of various isotypes. Alternatively, the antibody preparation may contain antibodies of only one isotype, or a number of selected isotypes.

For making a human(ized) monoclonal antibody, spleen cells are isolated from the immunized transgenic animal whose B-cells expressing the animal's endogenous immunoglobulin have been depleted. Isolated spleen cells are used either in cell fusion with transformed cell lines for the production of hybridomas, or cDNAs encoding antibodies are cloned by standard molecular biology techniques and expressed in transfected cells. The procedures for making monoclonal antibodies are well established in the art. See, e.g., European Patent Application 0 583 980 A1 (“Method For Generating Monoclonal Antibodies From Rabbits”), U.S. Pat. No. 4,977,081 (“Stable Rabbit-Mouse Hybridomas And Secretion Products Thereof”), WO 97/16537 (“Stable Chicken B-cell Line And Method of Use Thereof”), and EP 0 491 057 B1 (“Hybridoma Which Produces Avian Specific Immunoglobulin G”), the disclosures of which are incorporated herein by reference. In vitro production of monoclonal antibodies from cloned cDNA molecules has been described by Andris-Widhopf et al., “Methods for the generation of chicken monoclonal antibody fragments by phage display”, J Immunol Methods 242:159 (2000), and by Burton, D. R., “Phage display”, Immunotechnology 1:87 (1995), the disclosures of which are incorporated herein by reference.

In most instances the antibody preparation consists of unmodified immunoglobulins, i.e., human(ized) antibodies prepared from the animal without additional modification, e.g., by chemicals or enzymes. Alternatively, the immunoglobulin fraction may be subject to treatment such as enzymatic digestion (e.g., with pepsin, papain, plasmin, glycosidases, nucleases, etc.), heating, etc, and/or further fractionated.

Preferred embodiments of the invention are directed to methods for the suppression of endogenous immunoglobulin production in transgenic non-human animals producing humanized antibodies, allowing for the enrichment of desired human(ized) immunoglobulin. In one embodiment, transgenes comprising human(ized) immunoglobulin genes, a self-cleaving peptide and a recombinase are introduced into the transgenic animal using methods known in the art, ensuring concomitant expression of the human(ized) immunoglobulin and recombinase genes in B-cells, referred to as exogenous B-cells. Any variety of recombinases, self-cleaving peptides or immunoglobulin genes described herein or well-known in the art can be used in the transgene. Further in this embodiment, suppression of endogenous immunoglobulin production is achieved by selectively expressing a suicide gene in B-cells that express endogenous immunoglobulin, and therefore, are depleted due to cell death. Correspondingly, the suicide gene is excised out of the genome of exogenous B-cells via a recombinase-mediated mechanism due to expression of the transgene. Thus, exogenous B-cells survive and productively produce the transgene encoded human(ized) immunoglobulins. Different suicide genes described previously and those known in the art are embodiments of this invention. In one aspect of this embodiment, suicide genes are introduced via transgenes into the genome of the transgenic animal and their expression is driven, by an immune cell specific promoter, preferably, by a B-cell specific promoter, to selectively express suicide genes in B-cells thus preventing unnecessary cell death of non-B-cell populations. Various immune cell- and B-cell-specific promoters described herein or well-known in the art can be used to selectively express suicide genes in B-cells. In another embodiment, the transgenic animals used in the invention are gene converting animals or can undergo antibody diversification by gene rearrangement that stops early in life. Further, transgenic vectors and the transgenic animals generated using the methods described above also are embodiments of the invention.

The invention is further illustrated, but by no means limited, by the following examples.

### EXAMPLE 1

Construction of a Floxed DT-A Expression Vector

To achieve B-cell specific expression of the DT-A suicide gene The BAC clone 179L1 (Genebank Acc. No. AY495827) coding for rabbit kappa 1 is modified. A 46 kb fragment of the BAC clone comprising the kappa1 locus from the spacer down of V1 through the J locus, the intronic enhancer, the exon coding for the constant region, the 3 ′enhancer to the sequence downstream of the 3′enhancer is subcloned by ET-cloning. A pBELOBAC vector backbone with an additional gentamycin selection cassette is PCR amplified with primers having 50 bp homology to BAC 179L1. The forward primer additionally has an AttB integrase recognition site and a PvuI restriction enzyme recognition site. The reverse primer additionally has a PvuI restriction enzyme recognition site.

| Primer for Amplification of Modified pBELOBAC 11 | | |
|---|---|---|
| Forward | SEQ ID NO: 1 | 5′GGACCAGTTTACAATCCCACCTGCCATCTAAGAAA |
| | | GCTGGTCTCATCGTGGTGCCAGGGCGTGCCCTTGGGCTGGG |
| | | GGCGCGCGATCGGTCATAGCTGTTTCCTGTGTGAA 3′ |
| Reverse | SEQ ID NO: 2 | 5′ACTTTTGCAGGTAGAGGGGTTTGTCTGTAGGGAAAT |
| | | TCTGAACAATCATACGATCGAAGATGCGTGATCTGATCCTTC |
| | | AACTGA 3′ |
| Synthetic DT-A Fragment | | |
| | SEQ ID NO: 3 | cataattggacaaactacctacagagatttaaagctctaaggtaaatataaaatttttaagtg |
| | | tataatgtgttaaactactgattcctaattgtttgtgtattttagattccaacctatggaactgatgaatggg |
| | | agcagtggtggaatgcagatccactaggatctaacttgtttattgcagcttataatggttacaaataaag |
| | | caatagcatcacaaatttcacaaataaagcatttttttcactgcattctagttgtggtttgtccaaactcatc |
| | | aatgtatcttatcatgtctggatcataacttcgtatagcatacattatacgaagttatagttcatttgaagttt |
| | | ttaaagtgaacctcagtgactttgggatgtgaactctccgagtagaagcatgcgcactgcaggtaaac |
| | | ttgtgcagccctggtctgagctggggcagctggagacacagcccctgggctgagttctgagctgcc |
| | | ctgggcccttcagctgggcacagccctgccccgcccctgctcatttgcatgtccccagagcaccacc |
| | | cacctctctgggcatttaggagcaggctgctcccgccccatgcaggaggcagtgccaggcaggac |
| | | ccagcatggaccctgatgatgttgttgattcttctaaatcttttgtgatggaaaacttttcttcgtaccacg |
| | | ggactaaacctggttatgtagattccattcaaaaaggtatacaaaagccaaaatctggtacacaagga |
| | | aattatgacgatgattggaaagggttttatagtaccgacaataaatacgacgctgcgggatactctgta |
| | | gataatgaaaacccgctctctggaaaagctggaggcgtggtcaaagtgacgtatccaggactgacg |
| | | aaggttctcgcactaaaagtggataatgccgaaactattaagaaagagttaggtttaagtctcactgaa |
| | | ccgttgatggagcaagtcggaacggaagagtttatcaaaaggttcggtgatggtgcttcgcgtgtagt |
| | | gctcagccttcccttcgctgaggggagttctagcgttgaatatattaataactgggaacaggcgaaag |
| | | cgttaagcgtagaacttgagattaattttgaaacccgtggaaaacgtggccaagatgcgatgtatgag |
| | | tatatggctcaagcctgtgcaggaaatcgtgtcaggcgatctctttgacataattggacaaactaccta |
| | | cagagatttaaagctctaaggtaaatataaaatttttaagtgtataatgtgttaaactactgattcctaattg |
| | | tttgtgtattttagattccaacctatggaactgatgaatgggagcagtggtggaatgcagatccactag |
| | | gatctaacttgtttattgcagcttataatggttacaaataaagcaatagcatcacaaatttcacaaataaa |
| | | gcatttttttcactgcattctagttgtggtttgtccaaactcatcaatgtatcttatcatgtctggatcgaagt |
| | | tcctattccgaagttcctattctctagaaagtataggaacttcataacttcgtatagcatacattatacgaa |
| | | gttat |
| Primer for amplification of synthetic DT-A fragment | | |
| Forward | SEQ ID NO: 4 | 5′acataaatatactgtcttccaggatcttagagctcacctaaggaaacaagcataattgg |
| | | acaaactacctacagag 3′ |
| Reverse | SEQ ID NO: 5 | 5′aaacctactcatttacccacttttataaaatttctacttaaaagtgaatcataacttcgtata |
| | | atgtatgctatacg3′ |

For ET cloning, the PCR product is transformed into a streptomycin resistant E. coli strain containing the BAC 179L1 and the inducible lambda phage recombination enzymes Redα, Redβ and γ. These recombination proteins are expressed either from a cotransfected plasmid (DH10B E. coli cells with plasmid pSC101-γβα) or from a genomic integrated lambda prophage (DY380 E. coli strain). Positive clones 179L1(46 kb) are selected using gentamycin and verified by restriction enzyme digests.

A DNA fragment is synthesized chemically consisting of from 5′to 3′ two SV40 polyA sites—loxP site—rabbit kappa1 promoter—DT-A—FRT site—loxP site—two SV40 polyA sites. A kanamycin selection cassette is introduced into the FRT-site by FLP-mediated integration. The synthetic fragment is PCR amplified with primers having 50 bp homology to the subcloned 46 kb fragment of 179L1(46 kb). The J locus is changed against the synthetic DT-A fragment by ET-cloning. Positive clones are selected using kanamycin and verified by restriction enzyme analysis.

The kanamycin selection cassette is removed again by FLP-mediated recombination. Positive clones are selected by loss of the kanamycin resistance and verified by restriction enzyme digests and sequencing.

The final construct comprises of the the spacer down of V1, the synthetic DT-A fragment, the intronic enhancer, the exon coding for the constant region, the 3′enhancer to the sequence downstream of the 3′enhancer.

The construct is used for the generation of transgenic animals.

### EXAMPLE 2

Construction of a Human(ized) Heavy Chain Locus Encoding a Fusion Protein Consisting of the Membrane Form of IgM, a 2A Self-Cleaving Peptide, Cre-Recombinase

BAC and fosmid clones containing rabbit immunoglobulin heavy chain locus sequences were isolated from genomic DNA libraries using probes specific for the constant, variable, and joining gene segments or the 3′ enhancer region. Isolated BACs 27N5 (Genebank Acc. No. AY386696), 219D23 (Genebank Acc. No. AY386695), 225P18 (Genebank Acc. No. AY386697), 38A2 (Genebank Acc. No. AY386694) and fosmid Fos15B (Genebank Acc. No. AY3866968) were sequenced (Ros et al., Gene 330, 49-59.

Selected immunoglobulin coding sequences were exchanged with corresponding human counterparts by homologous recombination in E. Coli by ET cloning (E-Chiang Lee et al., Genomics 73, 56-65; Daiguan Yu et al., PNAS 97, 5978-5983; Muyrers et al., Nucleic Acids Research 27, 1555-1557; Zhang et al., Nature Biotechnology 18, 1314-1317).

Alternatively, DNA fragments were recombined by ligation in vitro and subsequent transformation of E. coli. BACs and/or Fos15B or parts thereof were combined by in vitro ligation and transformation, ET cloning, or by Cre recombinase mediated integration.

For ET cloning, vectors containing target sequence were transformed into a streptomycin resistant E. coli strain containing the inducible lambda phage recombination enzymes Redα, Redβ and γ. These recombination proteins were expressed either from a cotransfected plasmid (DH10B E. coli cells with plasmid pSC101-γβα) or from a genomic integrated lambda prophage (DY380 E. coli strain). The ET cloning procedure encompassed two homologous recombination steps.

In a first step the target locus was replaced by a selection—counter selection cassette (e.g. neo-rpsL which confers resistance to neomycin (neo) and sensitivity to streptomycin (rpsL). After isolation of neo-resistant colonies, insertion of the selection cassette by homologous recombination was confirmed by restriction enzyme analysis and partial sequencing.

In a second step, the rpsL-neo selection cassette was exchanged with a new sequence. Streptomycin resistant clones were analyzed by restriction analysis and sequencing. Fragments used for the ET cloning procedure had flanking sequences of 20 to 50 bp length, which were identical to target sequences. Sequences used for ligation had appropriate restriction enzyme sites at their 3′and 5′ends. These sites were either naturally occurring sites or they were introduced by PCR using primers containing appropriate sites.

Alternatively, sequences were generated synthetically.

A humanized heavy chain was constructed by replacement of rabbit J_{H}, Cμ in BAC 219D23 and Cγ in BAC 27N5 with their corresponding human counterparts by ET cloning. Human sequences used for the ET cloning procedures were amplified by PCR from human genomic DNA.

Human Cμ, Cγ and JH gene segments was amplified using primers with 50 bp homologies to the rabbit target sequences.

| | | |
|---|---|---|
| Region | SEQ ID Nos. | Sequence |

| | | |
|---|---|---|
| Cm | SEQ ID NO: 6 | 5′AAACAGCTTTTCACACCTCCCCTTTCTCT |
| | | CTTTGCTCCCCTGGGCCCTCAGGGAGTGCATCCG |
| | | CCCCAACCCTTTTCC3′ |
| Cm | SEQ ID NO: 7 | 5′CAGGGTTAGTTTGCATGCACACACACACA |
| | | GCGCCTGGTCACCCAGAGGGGTCAGTAGCAGGTG |
| | | CCAGCTGTGTCGGACATG3′ |
| Cg | SEQ ID NO: 8 | 5′GGTCAGGGGTCCTCCAGGGCAGGGGTCAC |
| | | ATTGTGCCCCTTCTCTTGCAGCCTCCACCAAGGG |
| | | CCCATCGGTC3′ |
| Cg | SEQ ID NO: 9 | 5′CACAGCTGCGGCGTGGGGGGGAGGGAGAG |
| | | GGCAGCTCGCCGGCACAGCGCTCATTTACCCGGA |
| | | GACAGGGAGAGGCTCTTC3′ |
| JH | SEQ ID NO: 10 | 5′GTGTTATAAAGGGAGACTGAGGGGGCAGA |
| | | GGCTGTGCTACTGGTACCTGGCTGAATACTTCCA |
| | | GCACTGGGGCCAGG3′ |
| JH | SEQ ID NO: 11 | 5′GGCCACAGAAAAGAGGAGAGAATGAAGGC |
| | | CCCGGAGAGGCCGTTCCTACCTGAGGAGACGGTG |
| | | ACCGTGGTCCCT TG-3′ |

After ligation of BAC clone 225P18 with clone 219D23 and BAC 27N5 with Fosmid 15B, the ligated constructs were transformation into E. coli and connected by Cre recombinase mediated insertion. This resulted in a functional locus consisting of 18 rabbit variable genes, rabbit D region, human J region, human Cμ, human Cγ, rabbit Cε, rabbit Cα4 and the 3′enhancer element.

A DNA fragment is synthesized chemically comprising of the coding sequence of the self-cleaving F2A peptide and the codon optimised coding sequence of the CRE recombinase (iCRE).

| | |
|---|---|
| SEQ ID Nos. | Synthetic F2A-iCre Fragment |

| | |
|---|---|
| SEQ ID NO: 12 | gtgaagcagactttgaattttgaccttctcaagttggcgggagacgtggagtc |
| | caacccagggcccatggtgcccaagaagaagaggaaagtctccaacctgctgactgtg |
| | caccaaaacctgcctgccctccctgtggatgccacctctgatgaagtcaggaagaacctg |
| | atggacatgttcagggacaggcaggccttctctgaacacacctggaagatgctcctgtct |
| | gtgtgcagatcctgggctgcctggtgcaagctgaacaacaggaaatggttccctgctgaa |
| | cctgaggatgtgagggactacctcctgtacctgcaagccagaggcctggctgtgaagac |
| | catccaacagcacctgggccagctcaacatgctgcacaggagatctggcctgcctcgcc |
| | cttctgactccaatgctgtgtccctggtgatgaggagaatcagaaaggagaatgtggatg |
| | ctggggagagagccaagcaggccctggcctttgaacgcactgactttgaccaagtcaga |
| | tccctgatggagaactctgacagatgccaggacatcaggaacctggccttcctgggcatt |
| | gcctacaacaccctgctgcgcattgccgaaattgccagaatcagagtgaaggacatctcc |
| | cgcaccgatggtgggagaatgctgatccacattggcaggaccaagaccctggtgtccac |
| | agctggtgtggagaaggccctgtccctgggggttaccaagctggtggagagatggatct |
| | ctgctggtgtggctgatgaccccaacaactacctgttctgccgggtcagaaagaatgg |
| | tgtggctgccccttctgccacctcccaactgtccacccgggccctggaagggatctttga |
| | ggccacccaccgcctgatctatggtgccaaggatgactctgggcagagatacctggcct |
| | ggtctggccactctgccagagtgggtgctgccagggacatggccagggctggtgtgtcc |
| | atccctgaaatcatgcaggctggtggctggaccaatgtgaacatagtgatgaactacatca |
| | gaaacctggactctgagactggggccatggtgaggctgctcgaggatggggactga |

Primers for Amplification of the F2A-iCRE Fragment

| | | |
|---|---|---|
| Forward | SEQ ID NO: 13 | 5′tccattcccaacacatgaacagcatctcacgccacctctgttgcctgcagg |
| | | tgaagcagactttgaattttgaccttc-3′ |
| Reverse | SEQ ID NO: 14 | 5′cagggccacggcgggcttgtctcttggcctccgacatccttctcaggtcat |
| | | cagtccccatcctcgagcagcctcacc-3′ |

The M2 membrane exon of IgM is changed against the synthetic M2-F2A-iCRE fragment by ET cloning.

The ET cloning procedure encompassed two homologous recombination steps. In a first step the target locus is replaced by a selection—counter selection cassette (e.g. neo-rpsL which confers resistance to neomycin (neo) and sensitivity to streptomycin (rpsL). For this the rpsL-neo selection cassette is PCR amplified with primers having 50 bp homology to the target locus. After isolation of neo-resistant colonies, insertion of the selection cassette by homologous recombination is confirmed by restriction enzyme analysis and partial sequencing.

In a second step, the rpsL-neo selection cassette is exchanged with the synthetic M2-F2A-iCRE fragment. Positive clones are identified by streptomycin resistance and are analyzed by restriction analysis and sequencing.

The resulting BAC is used for the generation of transgenic animals.

### EXAMPLE 3

Construction of a Humanized Light Chain Locus

Screening of a rabbit genomic BAC libraries resulted in the identification of two BACs (179L1 and 215M22) containing rabbit light chain K1 gene segments (Genebank accession numbers AY495827, AY495826).

Rabbit Cκ1 was exchanged with human Cκ allotype Km3 by ET cloning as described above.

Human Cκ (allotype Km3) was amplified by PCR with primers with 50 bp flanking sequences homologous to the target sequence.

| | |
|---|---|
| Region | Sequence |

| | | |
|---|---|---|
| Ck Km3 | SEQ ID NO: 15 | 5′GATGTCCACTGGTACCTAAGCCTCG |
| | | CCCTCTGTGCTTCTTCCCTCCTCAGGAACTG |
| | | TGGCTGCACCATCTGTCTTC3′ |
| Ck Km3 | SEQ ID NO: 16 | 5′GAGGCTGGGCCTCAGGGTCGCTGGC |
| | | GGTGCCCTGGCAGGCGTCTCGCTCTAACACT |
| | | CTCCCCTGTTGAAGCTCTTTGTG3 |

Homology arms were designed based on the published sequence of rabbit germline kappa (b5; GenBank Accession No. K01363) and matched the intron-exon boundary of Cκ.

The exchange of rabbit Cκ against the human Cκ in BAC 179L1 was verified by sequencing.

BAC 179L1-huCk was modified by two ET cloning. A neomycin selection cassette was amplified with primers having 50 bp homology to BAC 179L1. The forward primer additionally had an i-CeuI meganuclease site. The PCR product was used for ET cloning. Positive clones were selected with neomycin and checked for correctness by restriction enzyme digests and sequencing. A zeocin selection cassette was amplified with primers containing 50 bp sequences homologous to BAC 179L1. The forward primer additionally had an i-SceI meganuclease site. The PCR product was used for ET cloning. Positive clones were selected with zeozin and checked for correctness by restriction enzyme digests and sequencing.

BAC 215M22 was modified by one ET cloning. A gentamycin resistance gene was amplified with primers having 50 bp homology to BAC215M22. The forward primer additionally had an i-CeuI Meganuclease site and the reverse primer an i-SceI meganuclease site. The PCR product was used for ET cloning. Resulting clones were selected with gentamycin and checked for correctness by restriction enzyme digests and sequencing.

Amplification of Neomycin Resistance Gene

| | | |
|---|---|---|
| Forward | SEQ ID NO: 17 | 5′CTTTCTCTGTCCTTCCTGTGCGACGGT |
| | | TACGCCGCTCCATGAGCTTATCGTAACTA |
| | | TAACGGTCCTAAGGTAGCGATGGACAGCA |
| | | AGCGAACCGGA-3 |
| Reverse | SEQ ID NO: 18 | 5′GGACCAGTTTACAATCCCACCTGCCAT |
| | | CTAAGAAAGCTGGTCTCATCGTGTCAGAA |
| | | GAACTCGTCAAGAAG-3 |

Amplification of Zeocin Resistance Gene

| | | |
|---|---|---|
| Forward | SEQ ID NO: 19 | 5′CCCCCCCCGCCACTTCTCTTCTGTTTC |
| | | GTTTAAGTTCTACACTGACATACTAGGGA |
| | | TAACAGGGTAATAACGTTTACAATTTCGC |
| | | CTGATG-3 |
| Reverse | SEQ ID NO: 20 | 5′AGTGGGTAGGCCTGGCGGCCGCCTGGC |
| | | CGTCGACATTTAGGTGACACTATAGAAGG |
| | | ATCCTAGCACGTGTCAGTCCTGCT-3′ |

Amplification of Gentamycin Resistance Gene

| | | |
|---|---|---|
| Forward | SEQ ID NO: 21 | 5′TTACGCCAAGCTATTTAGGTGACACTA |
| | | TAGAATACTCAAGCTTTGATTGCTAACTA |
| | | TAACGGTCCTAAGGTAGCGATGAAGGCAC |
| | | GAACCCAGTTG-3′ |
| Reverse | SEQ ID NO: 22 | 5′GCGGAATTCTATGTCTAGTGGAGGGTG |
| | | AAGCTGGTGATTATAGAGTGAAAATTACC |
| | | CTGTTATCCCTATCGGCTTGAACGAATTG |
| | | TTAG-3′ |

Modified BAC179L1 and 225M22 were cut with i-CeuI and i-SceI. Fragments of 98 kb and 132 kb were purified and ligated. Resulting clones were selected with kanamycin and chloramphenicol and checked for correctness by restriction enzyme digests, PCR of the regions containing i-SceI and i-CeuI restriction sites, and sequencing. The resulting BAC was termed 179-215-huCk.

Rabbit Jk1 and Jk2 of BAC 179-215-huCk were replaced by ET cloning with a synthetic human rearranged kappa 1 VJ gene. A DNA fragment with rabbit promoter, rabbit leader, rabbit intron and human VJ gene was synthesized chemically. The codon usage of the synthetic human VJ was optimised to achieve highest DNA sequence homology to rabbit V kappa genes.

The synthetic human VJ was PCR amplified with a forward primer having 50 bp homology to BAC 179L1 and a revere primer having a homology to the gentamycin resistance gene and a FRT site. A gentamycin resistance gene was amplified with a forward primer having a FRT site and a reverse primer with 50 bp homology to BAC 179L1 and a FRT site. The human synthetic human VJ and the gentamycin resistance gene were combined by overlap extension PCR using the forward primer for the synthetic human VJ gene and the reverse primer for the gentamycin resistance gene. The resulting fragment was used for ET cloning. Positive clones were selected with gentamycin and checked for correctness by restriction enzyme digests and sequencing.

Primer for Amplification of Synthetic Human VJ

| | | |
|---|---|---|
| Forward | SEQ ID NO: 23 | 5′CATAAATATACTGTCTTCCAGGA |
| | | TCTTAGAGCTCACCTAAGGAAACAAGAGT |
| | | TCATTTGAAGTTTTTAAAGTG-3′ |
| Reverse | SEQ ID NO: 24 | 5′ACTCCAGAAGTTCCTATACTTTC |
| | | TAGAGAATAGGAACTTCGGAATAGGAACT |
| | | TCCTTTGATCTCCACCTTGGTC-3′ |

Primer for Amplification of Gentamycin
Resistance Gene

| | | |
|---|---|---|
| Forward | SEQ ID NO: 25 | 5′GAAGTTCCTATTCCGAAGTTCCT |
| | | ATTCTCTAGAAAGTATAGGAACTTCTGGA |
| | | GTTGTAGATCCTCTACG-3′ |
| Reverse | SEQ ID NO: 26 | 5′AAAACAAACCAATCAGGCAGAAACGGT |
| | | GAGGAATCAGTGAAACGGCCACTTACGAA |
| | | GTTCCTATACTTTCTAGAGAATAGGAACT |
| | | TCGGAATAGGAACTTCAAGATGCGTGATC |
| | | TGATCC-3′ |

The gentamycin resistance gene was removed by site specific recombination through expression of Flp recombinase. After recombination one FRT was left. The FRT site was deleted by ET cloning. A 232 bp fragment from the synthetic human VJ was amplified by PCR and used for ET cloning. Resulting colonies were screened by PCR for loss of the FRT site and confirmed by sequencing.

The neomycin resistance gene of BAC 179-215-huCk was replaced by ET cloning. A gentamycin resistance (pRep-Genta; Genebridges) gene was amplified by PCR with primers having 50 bp homology to BAC 179-215-huCk. The forward primer additionally had a loxP site, an attB site and a PvuI restriction site. Resulting clones were selected with gentamycin and checked for correctness by restriction enzyme digests and sequencing.

The resulting BAC was used for the generation of transgenic animals.

Primer for Deletion of FRT Site

| | | |
|---|---|---|
| Forward | SEQ ID NO: 27 | 5′TTATGCTGCATCCAGTTTGC-3′ |
| Reverse | SEQ ID NO: 28 | 5′AAAACAAACCAATCAGGCAG-3′ |

Primer for Screening

| | | |
|---|---|---|
| Forward | SEQ ID NO: 29 | 5′TGTGACATCCAGATGAC-3′ |
| Reverse | SEQ ID NO: 30 | 5′AAAACAAACCAATCAGGCAG-3 |

Primer for Amplification of
Gentamycin Resistance Gene

| | | |
|---|---|---|
| Forward | SEQ ID NO: 31 | 5′GGACCAGTTTACAATCCCACCTG |
| | | CCATCTAAGAAAGCTGGTCTCATCGTGGT |
| | | GCCAGGGCGTGCCCTTGGGCTGGGGGCGC |
| | | GATAACTTCGTATAGCATACATTATACGA |
| | | AGTTATCGATCGTGGAGTTGTAGATCC |
| | | TCTACG-3′ |
| Reverse | SEQ ID NO: 32 | 5′TTACGCCAAGCTATTTAGGTGAC |
| | | ACTATAGAATACTCAAGCTTTGATTGCAA |
| | | GATGCGTGATCTGATCCT-3′ |

### EXAMPLE 4

Generation of Transgenic Mice and Rabbits Expressing Humanized Heavy Chain Immunoglobulins

Transgenic rabbits and mice containing humanized heavy and light chain immunoglobulin loci and a floxed diphtheria toxin A gene under the control of the kappa light chain promoter/enhancer sequences are generated by injection of DNA into the pronuclei of fertilized oocytes and subsequent transfer of embryos into foster mothers. Transgenic founder animals are identified by PCR. Expression of human(ized) immunoglobulin M and G is measured by ELISA. Expression of humanized IgG is 1-5 mg/ml. Expression of mouse and rabbit IgG is 1-5 ug/ml, respectively.

### EXAMPLE 5

Generation of Transgenic Chicken Expressing Humanized Heavy Chain Immunoglobulins

Transgenic chicken are generated by testis mediated gene transfer. DNA constructs (50 ug) are mixed with 250 ul lipofection reagent (superfect) in 500 ul 0.9% NaCl and injected in the testis of roosters. Three to four weeks later roosters with transgenic sperm are identified by PCR analysis and mated with hens. Transgenic offspring are identified by PCR. Expression of humanized IgG is 1-5 mg/ml. Expression of chicken IgY is 1-5 ug/ml.

All references cited throughout the disclosure along with references cited therein are hereby expressly incorporated by reference.

While the invention is illustrated by reference to certain embodiments, it is not so limited. One skilled in the art will understand that various modifications are readily available and can be performed without substantial change in the way the invention works. All such modifications are specifically intended to be within the scope of the invention claimed herein.

What is claimed is:

1. A method for selective suppression of endogenous immunoglobulin production in B-cells of a non-human transgenic animal carrying an exogenous immunoglobulin translocus, comprising selectively expressing at least one suicide gene in B-cells producing an endogenous immunoglobulin of said non-human transgenic animal, but not in B cells producing an exogenous immunoglobulin, whereby B cells producing the endogenous immunoglobulin are depleted, and production of the endogenous immunoglobulin is suppressed, without suppressing the production of said exogenous immunoglobulin.

2. The method of claim 1 wherein said exogenous immunoglobulin is a human(ized) immunoglobulin heavy and/or light chain sequence.

3. The method of claim 2 wherein said suicide gene, introduced into the B-cells of said non-human transgenic animal, is under the control of a B-cell specific promoter and is flanked by recombination sequences.

4. The method of claim 3 wherein said human(ized) immunoglobulin chain translocus is introduced into the B-cells of said non-human transgenic animal as part of an expression construct, additionally encoding a recombinase recognizing said recombination sequences, wherein expression of said suicide gene is inactivated through expression of said recombinase in B-cells expressing said human(ized) immunoglobulin translocus.

5. The method of claim 1 wherein said suicide gene is selected from the group consisting of bacterial, fungal, insecticidal and plant toxins.

6. The method of claim 1 wherein said suicide gene is a diphtheria toxin chain A.

7. The method of claim 1 wherein said suicide gene is a prodrug converting enzyme.

8. The method of claim 7 wherein the prodrug converting enzyme is of non-mammalian origin.

9. The method of claim 8 wherein said prodrug converting enzyme of non-mammalian origin is selected from the group consisting of viral thymidine kinase (TK), bacterial cytosine deaminase (CD), bacterial carboxypeptidase G2 (CPG2), purine nucleotide phosphorylase (PNP), thymidine phosphorylase (TP), nitroreductase (NR), D-amino acid oxidase (DAAO), xanthine-guanine phosphoribosyl transferease (XGPRT), penicillin-G amidase (PGA), β-lactamase, multiple drug activation enzyme (MDAE), β-galactosidase (β-Gal), horseradish peroxidase (HRP) and deoxyribonucleotide kinase (DRNK).

10. The method of claim 7 wherein the prodrug converting enzyme is of human origin.

11. The method of claim 10 wherein the prodrug converting enzyme of human origin is selected from the group consisting of deoxycytidine kinase (dCK), carboxyesterases (CEs), carboxypeptidase A (CPA), β-glucuronidase (-Glu), and cytochrome P450 (CYP).

12. The method of claim 4 wherein said recombinase is selected from the group consisting of a Cre, Cre-like, Flp, φC31, λ integrase, phage R4 recombinase, TP901-1 recombinase, a prokaryotic transposase, a eukaryotic transposase, a viral retrotransposase, a Drosophila copia-like retrotransposase and a non-viral retrotransposase.

13. The method of claim 12 wherein said transposase or retrotransposase is selected from the group consisting of Tn1, Tn2, Tn3, Tn4, Tn5, Tn6, Tn9, Tn10, Tn30, Tn101, Tn501, Tn903, Tn1000, Tn1681, Tn2901, Drosophila mariner, sleeping beauty transposase, Drosophila P element, maize Ac, Ds, Mp, Spm, En, dotted, Mu, I, L1, Tol2 Tc1, Tc3, Mariner (Himar 1), Mariner (mos 1) and Minos.

14. The method of claim 1 wherein said non-human transgenic animal substantially stops antibody diversification by gene rearrangement early in life.

15. The method of claim 14 wherein said non-human transgenic animal substantially stops antibody diversification within the first month of its life.

16. The method of claim 1 wherein said non-human transgenic animal is selected from the group consisting of rodents, rabbits, birds, cows, pigs, sheep, goats and horses.

17. The method of claim 16 wherein said rodent is a mouse or a rat.

18. A transgenic expression construct comprising a first transgene further comprising a human or humanized immunoglobulin heavy and/or light chain translocus, a self-cleaving peptide and a recombinase.

19. A transgenic expression construct comprising a second transgene further comprising a suicide gene that is under the control of a B-cell specific promoter, and is flanked by recombination sites recognized by a recombinase.

20. A transgenic expression construct comprising:
a first transgene further comprising a human or humanized immunoglobulin heavy and/or light chain locus, a self-cleaving peptide and a recombinase, and,
a second transgene further comprising a suicide gene that is under the control of a B-cell specific promoter, and is flanked by recombination sites recognized by said recombinase.

21. The transgenic expression construct of claim 18, 19 or 20 wherein said recombinase is a site specific recombinase selected from the group consisting of a Cre, Cre-like, Flp, φC31, λ integrase, phage R4 and TP901-1 recombinase.

22. The transgenic expression construct of claim 18, 19 or 20 wherein said recombinase is either a prokaryotic or a eukaryotic transposase.

23. The transgenic expression construct of claim 22 wherein said recombinase is either a viral, Drosophila copia-like or non-viral retrotransposon.

24. The transgenic expression construct of claim 23 wherein said retrotransposon is selected from the group consisting of Tn1, Tn2, Tn3, Tn4, Tn5, Tn6, Tn9, Tn10, Tn30, Tn101, Tn501, Tn903, Tn1000, Tn1681, Tn2901, Drosophila mariner, sleeping beauty transposase, Drosophila P element, maize Ac, Ds, Mp, Spm, En, dotted, Mu, I, L1, Tol2 Tc1, Tc3, Mariner (Himar 1), Mariner (mos 1) and Minos.

25. The transgenic expression construct of claims 19 or 20 wherein said recombination sites are selected from a group consisting of a lox P site, FRT site, a bacterial genomic recombination site and a phage recombination site.

26. The transgenic expression construct of claim 25 wherein said bacterial genomic recombination site is attB and said phage recombination site is an attP or a pseudo-attP or a pseudo-attB site.

27. The transgenic expression construct of claims 18 or 20 wherein said self-cleaving peptide is obtained from viral 2A/2B or 2A-like/2B sequences.

28. The transgenic expression construct of claim 27 wherein said virus is selected from the group consisting of the picornaviridae virus family, the equine rhinitis A (ERAV) virus family, the picornavirus-like insect virus family or from the type C rotavirus family.

29. The transgenic expression construct of claim 27 wherein said virus is selected from the group consisting of the foot and mouth disease virus (FMDV), the equine rhinitis A (ERAV) virus, or the Thosea asigna virus (TaV).

30. The transgenic expression construct of claims 19 or 20 wherein said suicide gene is specifically expressed in B-cells using a promoter/enhancer selected from the group consisting of CD19, CD20, CD21, CD22, CD23, CD24, CD40, CD72, Blimp-1, CD79b, mb-1, tyrosine kinase blk, VpreB, immunoglobulin kappa light chain, immunoglobulin lambda-light chain and immunoglobulin J-chain or modifications thereof.

31. The transgenic expression construct of claim 30 wherein said B-cell specific promoter/enhancer is the kappa light chain gene promoter or modifications thereof.

32. A non-human transgenic animal expressing the transgenic expression constructs of claims 18, 19 or 20.

33. The non-human transgenic animal of claim 32 that generates antibody diversity substantially by gene conversion.

34. The non-human transgenic animal of claim 32 that is selected from the group consisting of rodents, rabbits, birds including chickens, turkeys, ducks and geese.

35. The non-human transgenic animal of claim 34 that is either a mouse or a rat.


SEQUENCE LISTING (32 sequences)

SEQ ID NO: 1 - DNA, 111 bp, Artificial Sequence
  Forward Primer
ggaccagtttacaatcccacctgccatctaagaaagctggtctcatcgtggtgccagggcgtgcccttgggctgggggcgcgcgatcggtcatagctgtttcctgtgtgaa

SEQ ID NO: 2 - DNA, 84 bp, Artificial Sequence
  Reverse Primer
acttttgcaggtagaggggtttgtctgtagggaaattctgaacaatcatacgatcgaagatgcgtgatctgatccttcaactca

SEQ ID NO: 3 - DNA, 1600 bp, Artificial Sequence
  Synthetic Sequence
cataattggacaaactacctacagagatttaaagctctaaggtaaatataaaatttttaagtgtataatgtgttaaactactgattcctaattgtttgtgtattttagattccaacctatggaactgatgaatgggagcagtggtggaatgcagatccactaggatctaacttgtttattgcagcttataatggttacaaataaagcaatagcatcacaaatttcacaaataaagcatttttttcactgcattctagttgtggtttgtccaaactcatcaatgtatcttatcatgtctggatcataacttcgtatagcatacattatacgaagttatagttcatttgaagtttttaaagtgaacctcagtgactttgggatgtgaactctccgagtagaagcatgcgcactgcaggtaaacttgtgcagccctggtctgagctggggcagctggagacacagcccctgggctgagttctgagctgccctgggcccttcagctgggcacagccctgccccgcccctgctcatttgcatgtccccagagcaccacccacctctctgggcatttaggagcaggctgctcccgccccatgcaggaggcagtgccaggcaggacccagcatggaccctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgacataattggacaaactacctacagagatttaaagctctaaggtaaatataaaatttttaagtgtataatgtgttaaactactgattcctaattgtttgtgtattttagattccaacctatggaactgatgaatgggagcagtggtggaatgcagatccactaggatctaacttgtttattgcagcttataatggttacaaataaagcaatagcatcacaaatttcacaaataaagcatttttttcactgcattctagttgtggtttgtccaaactcatcaatgtatcttatcatgtctggatcgaagttcctattccgaagttcctattctctagaaagtataggaacttcataacttcgtatagcatacattatacgaagttat

SEQ ID NO: 4 - DNA, 76 bp, Artificial Sequence
  Forward Primer
acataaatatactgtcttccaggatcttagagctcacctaaggaaacaagcataattggacaaactacctacagag

SEQ ID NO: 5 - DNA, 77 bp, Artificial Sequence
  Reverse Primer
aaacctactcatttacccacttttataaaatttctacttaaaagtgaatcataacttcgtataatgtatgctatacg

SEQ ID NO: 6 - DNA, 78 bp, Artificial Sequence
  Primer
aaacagcttttcacacctcccctttctctctttgctcccctgggccctcagggagtgcatccgccccaacccttttcc

SEQ ID NO: 7 - DNA, 81 bp, Artificial Sequence
  Primer
cagggttagtttgcatgcacacacacacagcgcctggtcacccagaggggtcagtagcaggtgccagctgtgtcggacatg

SEQ ID NO: 8 - DNA, 73 bp, Artificial Sequence
  Primer
ggtcaggggtcctccagggcaggggtcacattgtgccccttctcttgcagcctccaccaagggcccatcggtc

SEQ ID NO: 9 - DNA, 81 bp, Artificial Sequence
  Primer
cacagctgcggcgtgggggggagggagagggcagctcgccggcacagcgctcatttacccggagacagggagaggctcttc

SEQ ID NO: 10 - DNA, 77 bp, Artificial Sequence
  Primer
gtgttataaagggagactgagggggcagaggctgtgctactggtacctggctgaatacttccagcactggggccagg

SEQ ID NO: 11 - DNA, 77 bp, Artificial Sequence
  Primer
ggccacagaaaagaggagagaatgaaggccccggagaggccgttcctacctgaggagacggtgaccgtggtcccttg

SEQ ID NO: 12 - DNA, 1122 bp, Artificial Sequence
  Synthetic Fragments
gtgaagcagactttgaattttgaccttctcaagttggcgggagacgtggagtccaacccagggcccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacatagtgatgaactacatcagaaacctggactctgagactggggccatggtgaggctgctcgaggatggggactga

SEQ ID NO: 13 - DNA, 78 bp, Artificial Sequence
  Synthetic Sequence
tccattcccaacacatgaacagcatctcacgccacctctgttgcctgcaggtgaagcagactttgaattttgaccttc

SEQ ID NO: 14 - DNA, 78 bp, Artificial Sequence
  Reverse Primer
cagggccacggcgggcttgtctcttggcctccgacatccttctcaggtcatcagtccccatcctcgagcagcctcacc

SEQ ID NO: 15 - DNA, 76 bp, Artificial Sequence
  Synthetic
gatgtccactggtacctaagcctcgccctctgtgcttcttccctcctcaggaactgtggctgcaccatctgtcttc

SEQ ID NO: 16 - DNA, 79 bp, Artificial Sequence
  Synthetic
gaggctgggcctcagggtcgctggcggtgccctggcaggcgtctcgctctaacactctcccctgttgaagctctttgtg

SEQ ID NO: 17 - DNA, 96 bp, Artificial Sequence
  Forward Primer
ctttctctgtccttcctgtgcgacggttacgccgctccatgagcttatcgtaactataacggtcctaaggtagcgatggacagcaagcgaaccgga

SEQ ID NO: 18 - DNA, 71 bp, Artificial Sequence
  Reverse Primer
ggaccagtttacaatcccacctgccatctaagaaagctggtctcatcgtgtcagaagaactcgtcaagaag

SEQ ID NO: 19 - DNA, 91 bp, Artificial Sequence
  Forward Primer
ccccccccgccacttctcttctgtttcgtttaagttctacactgacatactagggataacagggtaataacgtttacaatttcgcctgatg

SEQ ID NO: 20 - DNA, 80 bp, Artificial Sequence
  Reverse Primer
agtgggtaggcctggcggccgcctggccgtcgacatttaggtgacactatagaaggatcctagcacgtgtcagtcctgct

SEQ ID NO: 21 - DNA, 96 bp, Artificial Sequence
  Forward Primer
ttacgccaagctatttaggtgacactatagaatactcaagctttgattgctaactataacggtcctaaggtagcgatgaaggcacgaacccagttg

SEQ ID NO: 22 - DNA, 89 bp, Artificial Sequence
  Reverse Primer
gcggaattctatgtctagtggagggtgaagctggtgattatagagtgaaaattaccctgttatccctatcggcttgaacgaattgttag

SEQ ID NO: 23 - DNA, 73 bp, Artificial Sequence
  Forward Primer
cataaatatactgtcttccaggatcttagagctcacctaaggaaacaagagttcatttgaagtttttaaagtg

SEQ ID NO: 24 - DNA, 74 bp, Artificial Sequence
  Reverse Primer
actccagaagttcctatactttctagagaataggaacttcggaataggaacttcctttgatctccaccttggtc

SEQ ID NO: 25 - DNA, 69 bp, Artificial Sequence
  Forward Primer
gaagttcctattccgaagttcctattctctagaaagtataggaacttctggagttgtagatcctctacg

SEQ ID NO: 26 - DNA, 120 bp, Artificial Sequence
  Reverse Primer
aaaacaaaccaatcaggcagaaacggtgaggaatcagtgaaacggccacttacgaagttcctatactttctagagaataggaacttcggaataggaacttcaagatgcgtgatctgatcc

SEQ ID NO: 27 - DNA, 20 bp, Artificial Sequence
  Forward Primer
ttatgctgcatccagtttgc

SEQ ID NO: 28 - DNA, 20 bp, Artificial Sequence
  Reverse Primer
aaaacaaaccaatcaggcag

SEQ ID NO: 29 - DNA, 17 bp, Artificial Sequence
  Forward Primer
tgtgacatccagatgac

SEQ ID NO: 30 - DNA, 20 bp, Artificial Sequence
  Reverse Primer
aaaacaaaccaatcaggcag

SEQ ID NO: 31 - DNA, 143 bp, Artificial Sequence
  Forward Primer
ggaccagtttacaatcccacctgccatctaagaaagctggtctcatcgtggtgccagggcgtgcccttgggctgggggcgcgataacttcgtatagcatacattatacgaagttatcgatcgtggagttgtagatcctctacg

SEQ ID NO: 32 - DNA, 70 bp, Artificial Sequence
  Reverse Primer
ttacgccaagctatttaggtgacactatagaatactcaagctttgattgcaagatgcgtgatctgatcct